(19) United States
(12) Patent Application Publication
(10) Pub. No.: US 2025/0188479 A1
(43) Pub. Date: Jun. 12, 2025

(54) Biosynthesis Of Rose Aromas

(71) Applicant: AGENCY FOR SCIENCE, TECHNOLOGY AND RESEARCH, Singapore (SG)

(72) Inventors: Jian Ting Leonard Ong, Singapore (SG); Chin Chin Lim, Singapore (SG); Congqiang Simon Zhang, Singapore (SG); Sudha Devi D/O Manbahal Shukal, Singapore (SG); Xixian Chen, Singapore (SG); Rehka T, Singapore (SG)

(21) Appl. No.: 18/845,495

(22) PCT Filed: Mar. 10, 2023
(86) PCT No.: PCT/SG2023/050156

§ 371 Date: Sep. 10, 2024

(30) Foreign Application Priority Data
Mar. 10, 2022 (SG) 10202202455U
Nov. 7, 2022 (SG) 10202251656V

(51) Int. Cl. C12N 15/52; C12N 9/04; C12N 9/10; C12N 9/12; C12N 9/14; C12N 9/88; C12N 9/90; C12P 7/04; C12P 7/40

(52) U.S. Cl. CPC C12N 15/52; C12N 9/0006; C12N 9/1025; C12N 9/1029; C12N 9/1085; C12N 9/1205; C12N 9/1229; C12N 9/14; C12N 9/88; C12N 9/90; C12P 7/04; C12P 7/40; C12Y 101/01034; C12Y 203/01009; C12Y 203/01075; C12Y 203/0301; C12Y 205/0101; C12Y 205/01029; C12Y 207/01036; C12Y 207/04002; C12Y 401/01033; C12Y 503/03002; C07K 2319/00

(57) ABSTRACT

The present invention relates to host cells comprising genes of the mevalonate and Nudix pathways, engineered fusion proteins of enzymes of the mevalonate and Nudix pathways, methods as well as kits for producing geraniol and geranyl acetate.

### CROSS-REFERENCE TO RELATED APPLICATIONS

This application claims the benefit of priority of Singapore Application No. 10202251656V, filed 7 Nov. 2022 and Singapore Application No. 10202202455U, filed 10 Mar. 2022, the contents of it being hereby incorporated by reference in its entirety for all purposes.

### FIELD OF THE INVENTION

The invention relates to biosynthesis of terpenoids, in particular rose aroma molecules.

### BACKGROUND OF THE INVENTION

Rose oils are rich in volatile molecules, among them, monoterpenes play critical roles in characterizing rose scents. Geraniol, a monoterpene alcohol with rose-like odor and taste, is an important commercial flavor and fragrance molecule. Geraniol and its ester derivative, geranyl acetate, are the two most important monoterpenes in rose oils. Geraniol is widely used in deodorants, perfumes and cosmetic creams and is also an effective plant-based mosquito repellent and insecticide with low mammalian toxicity and biodegradability. Geranyl acetate, the ester derivative of geraniol, is also widely used in the cosmetic industry due to its floral and fruity scent.

However, these molecules are only produced in low concentrations in plants. Currently, geraniol and geranyl acetate are predominantly produced by chemical synthesis which is unsustainable and not environmentally friendly. In addition, there is an increasing demand among consumers for natural ingredients or bioingredients. Hence, there is growing interest in the development of biotechnological routes for the production of geraniol and geranyl acetate and in recent years, biotechnological routes for geraniol production have been developed. Most of these use plant geraniol synthases (GESs) which have inadequate solubility and/or activity in microbes that limit the yield of geraniol. Therefore, there is a need to provide a method and system for the biosynthesis of geraniol and geranyl acetate that overcomes, or at least ameliorates, one or more of the disadvantages described above.

### SUMMARY

In one aspect, provided herein is a host cell comprising one or more vectors comprising a polynucleotide sequence encoding: one or more genes of the mevalonate pathway; and one or more genes of the Nudix pathway.

In another aspect, provided herein is an engineered fusion protein comprising a diphosphate synthase or prenyltransferase of the mevalonate pathway and a nudix hydrolase; or a diphosphate synthase or prenyltransferase of the mevalonate pathway, a nudix hydrolase and a geranyl synthase enzyme (GES) of the terpene synthase pathway; or a diphosphate synthase or prenyltransferase of the mevalonate pathway and a GES of the terpene synthase pathway.

In another aspect, provided herein is a method of geraniol, geranyl acetate, or geraniol and geranyl acetate production comprising culturing the host cell as described herein in a culture medium, wherein the culture medium comprises an inducer and at least one carbon substrate.

In another aspect, provided herein is a kit for producing geraniol, geranyl acetate, or geraniol and geranyl acetate, wherein the kit comprises the host cell as described herein with instructions for use.

### Definitions

As used herein, the term “isoprenoid” refers to a large and diverse class of naturally-occurring class of organic compounds composed of two or more units of hydrocarbons, with each unit consisting of five carbon atoms arranged in a specific pattern.

As used herein, the term “monoterpene” or “monoterpenoids” are a class of isoprenoids produced from geranyl diphosphate by various monoterpene synthases. Monoterpenoids have two isoprenoid units. Monoterpenes are secondary metabolites in plants and the main constituents of essential oils, cosmetics, food flavorings, cleaning products and drugs. They contribute to the specific smell characters of plants. Monoterpenes are industrially used as flavour, fragrant, and cosmetic constituents. Moreover, they are precursors of several flavour compounds such as citronellol, geraniol, menthol, and verbenol.

As used herein, the term “mevalonate pathway” refers to a cellular metabolic pathway that plays a key role in multiple cellular processes by synthesizing sterol isoprenoids, such as cholesterol, and non-sterol isoprenoids, such as dolichol, heme-A, isopentenyl tRNA and ubiquinone. The mevalonate pathway is the first recognized pathway for biosynthesis of isopentenyl pyrophosphate (IPP) and dimethylallyl pyrophosphate (DMAPP), which involves a series of six enzymatic steps that convert acetyl-CoA to IPP. Three molecules of acetyl-CoA are condensed to synthesize mevalonate in the first two steps of the mevalonate pathway. The enzymes acetoacetyl-CoA thiolase and HMG-COA synthase (HMGS) catalyze the condensation reactions to form hydroxymethylglutaryl-CoA (HMG-COA). Furthermore, reduction of HMG-COA into mevalonate is catalyzed by HMG-CoA reductase (HMGR). The mevalonate thus synthesized is phosphorylated and decarboxylated to form IPP. The phosphorylation is first catalyzed by mevalonate kinase followed by the action of phosphomevalonate kinase (PMK) to form mevalonate-5-pyrophosphate. Decarboxylation is the last step, in which phosphomevalonate decarboxylase catalyzes the ATP-dependent decarboxylation of mevalonate-5-pyrophosphate to form IPP. IPP may interact with DHNA to form AQ or isomerases to form DMAPP by IPP isomerase (IDI). Genes of the mevalonate pathway refer to genes that encode enzymes of the mevalonate pathway.

As used herein, the term “nudix hydrolase” refers to a superfamily of hydrolytic enzymes and are found in all classes of organism. Nudix hydrolases hydrolyse a wide range of organic pyrophosphates, including nucleoside di- and triphosphates, dinucleoside and diphosphoinositol polyphosphates, nucleotide sugars and RNA caps, with varying degrees of substrate specificity.

As used herein, the term “Nudix pathway” refers to a metabolic pathway that involves a diphosphohydrolase belonging to the Nudix enzyme family. The cytosolic Nudix hydrolase (such as AtNUDX1, NudI, RhNUDX1) converts geranyl diphosphate (GPP) into geranyl monophosphate (GP), which is then hydrolyzed to geraniol by phosphatase activity. Genes of the Nudix pathway refer to genes that encode enzymes of the Nudix pathway.

As used herein, the term “geraniol” refers to an acyclic monoterpene alcohol with the formula C₁₀H₁₈O, 3,7-dimethyl-2,6-octadien-1-ol. Geraniol can be produced by aromatic plants. Geraniol can also be biosynthesized in engineered strains, including Saccharomyces cerevisiae.

As used herein, the term “geranyl acetate” is a monoterpene that is the acetate ester derivative of geraniol.

As used herein, the term “polypeptides” includes polypeptides, proteins, peptides, fragments of polypeptides, and fusion polypeptides.

As used herein, a “nucleic acid” refers to two or more deoxyribonucleotides and/or ribonucleotides covalently joined together in either single or double-stranded form.

As used herein, the term “operably linked” refers to a functional linkage between a nucleic acid expression control sequence (such as a promoter) and a second nucleic acid sequence, wherein the expression control sequence regulates the transcription of the nucleic acid corresponding to the second sequence.

As used herein, the term “variant” refers to a modification in the DNA sequence. The modification in the DNA sequence includes mutation, truncation, translocation, substitution, deletion and insertion, resulting in the alteration of the activity of the gene.

The term “promoter” as used herein refers to a region of the DNA that initiates transcription of a gene. The region of the DNA is typically located near the transcription start site of a gene and upstream on the DNA. A promoter may be inducible or non-inducible. The term “inducible promoter” as used herein refers to a promoter that can be regulated in the response to specific stimuli, also known as inducers. The promoter system may be modified to be inducible. Examples of inducible promoter systems include the Tet-on system, Tet-off system, T7 system, Trp system, Tac system, lambda cl857-PL system, bacterial EL222 system and Lac system. A promoter may also be a constitutive promoter which is a promoter that is always active.

The term “ribosomal binding site” as used herein in the context of the application refers to a site of an mRNA molecule which recruits and binds the ribosome, allowing the selection of the proper initiation codon during the initiation of translation. The ribosomal binding site controls the accuracy and efficiency of the initiation of mRNA translation.

As used herein, the term “linker” refers to short amino acid sequences that separate multiple domains in a recombinant or fusion protein. Linkers function to prohibit unwanted interactions between the discrete domains. However, there are flexbile Gly-rich linkers that connect various domains in a single protein without interfering with the function of each domain. Gly-rich linkers can also help create a covalent link between proteins to form a stable protein-protein complex. The lengths of linkers vary from 2 to 31 amino acids, optimized for each condition so that the linker does not impose any constraints on the conformation or interactions of the linked partners.

As used herein, the term “deficient” in the context of the expression of a gene or protein refers to a reduction in expression level of a gene or protein relative to a baseline level of expression of the gene or protein. Deficient in the context of the expression of a gene or protein may also refer to non-expression of a gene or protein in a scenario where the gene or protein would otherwise be expressed. The baseline expression of a gene or a protein would be understood to mean the expression level of an unmutated gene or a wild type gene, or in the context where the gene or protein would otherwise be expressed, the expression level of the gene or protein.

As used herein, the term “co-expressed” refers to transcription and/or translation of two or more genes as a single unit. The transcription and/or translation of two or more genes as a single unit may occur via fusion of two or more genes. Alternatively, co-expression in the context of the expression of genes and/or proteins may also refer to the transcription and/or translation of two or more genes as separate units.

As used herein, the term “about”, is used in the context of, but not limited to, concentrations of components and percentages of compounds, typically refers to +/−10% of the stated value, to +/−9% of the stated value, to +/−8% of the stated value, to +/−7% of the stated value, to +/−6% of the stated value, to +/−5% of the stated value, +/−4% of the stated value, more typically +/−3% of the stated value, more typically, +/−2% of the stated value, even more typically +/−1% of the stated value, and even more typically +/−0.5% of the stated value. Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE DRAWINGS

The invention will be better understood with reference to the detailed description when considered in conjunction with the non-limiting examples and the accompanying drawings, in which:

FIG. 1 depicts the biosynthetic pathway of geraniol and geranyl acetate. The biosynthetic pathway consists of 1) mevalonate pathway genes, including atoB, hmgS and thmgR, mevk, pmk, pmd and idi; 2) monoterpene pathway genes, including GPPS, RhNUDX1, phosphatase and RhAAT1; 3) other genes: yjgB. Abbreviation for the compounds: MVA, mevalonate; IPP, isopentenyl pyrophosphate; DMAPP, dimethylallyl pyrophosphate; GPP, geranyl pyrophosphate; GP, geranyl phosphate. Dashed arrow indicates multiple enzymatic steps. The genes expressed encode the following enzymes: GPPS, GPP synthase; RhNUDX1, the Nudix hydrolase from Rosa hybrida; RhAAT1, alcohol acyltransferase from Rosa hybrida that catalyze geraniol into geranyl acetate; yjgB, an alcohol dehydrogenase that converts geraniol into geranial.

FIG. 2 shows the in vitro characterization and in vivo application of RhNUDX1.

FIG. 2A shows the characterization of purified RhNUDX1 (calculated Kₘ and K_{cat} in Table 5).

FIG. 2B depicts the study of pH effect on RhNUDX1. FIG. 2C shows the in vivo application of RhNUDX1 to produce geraniol. FIG. 2D shows the OD₆₀₀ of different strains. Error bars, mean±s.d., n=4-7.

FIG. 3 depicts the enhancing GPP supply and pathway balancing. FIG. 3A shows the monoterpene titres and FIG. 3B displays the OD₆₀₀ of strains constructed by combining RBS engineering and GPPS screening method. FIG. 3C shows the geraniol titre and FIG. 3D depicts the OD₆₀₀ of strains constructed by pathway balancing. Error bars, mean±s.d., n=3-6.

FIG. 4 shows the fusion of GPPS and RhNUDX1. (A) The monoterpene titres and (B) OD₆₀₀ in strains with fused and free forms of GPPS and RhNUDX1.

FIG. 5 depicts YjgB deletion. (A) The monoterpene titres and (B) OD₆₀₀ in wildtype (WT) and mutant (ΔyjgB) strains.

FIG. 6 refers to abiotic strategies to minimize the by-product formation. FIG. 6A shows the GC chromatograms of chemical standards and our optimized (40 mM lactose) and control (20 mM lactose) condition. FIG. 6B depicts the monoterpene production and OD₆₀₀ in auto-induction defined media with lactose dosage tuning. FIG. 6C shows the monoterpene production and OD₆₀₀ in manually induced defined media with IPTG dosage tuning. Error bars, mean±s.d., n=2-3.

FIG. 7 shows the comparison of GES, RhNUDX1, NudI and their combination with ispA and AgGPPS. FIG. 7A shows the geraniol production and FIG. 7B shows the OD₆₀₀ in the strains using various enzymes. FIG. 7C shows geraniol production and FIG. 7D shows the OD 600 in the strains using combinations of GES/Nudix enzymes and additional GPPSs. Error bars, mean±s.d., n=2.

FIG. 8 refers to the optimization of geranyl acetate bioproduction. FIG. 8A shows the geraniol titre and OD₆₀₀ of strains constructed by pathway balancing. FIG. 8B shows the induction optimization of the two best strains of geranyl acetate. Error bars, mean±s.d., n=3.

FIG. 9 refers to the fed-batch fermentation of geraniol and geranyl acetate. FIG. 9A shows the time-course profiles of geraniol and OD₆₀₀. FIG. 9B shows the time-course profiles of geranyl acetate and OD₆₀₀.

FIG. 10 illustrates the deletion of ackA-pta on geranyl acetate production. Glycerol concentrations in media were 20 or 30 g/L, respectively. Here, the ‘-’ refers to the strain without the ackA-pta deletion. The cells were grown in 10 mL TB medium in 125 mL baffled flasks at 100 rpm, 28° C. for 3 days.

FIG. 11 depicts the tryptone supplementation on geraniol biosynthesis. Error bars, mean±s.d., n=2. The cells were grown in 1 mL chemically defined medium in tubes at 300 rpm, 28° C. for 3 days.

FIG. 12 shows the optimization of dodecane/medium ratio. Error bars, mean±s.d., n=2. The cells were grown in 10 mL chemically defined medium in 125 mL baffled flasks at 100 rpm, 28° C. for 3 days.

FIG. 13 illustrates the enzymatic combinations used in this study.

### DETAILED DESCRIPTION OF THE PRESENT INVENTION

In a first aspect, the present invention refers to a host cell comprising one or more vectors comprising a polynucleotide sequence encoding: a) one or more genes of the mevalonate pathway; and b) one or more genes of the Nudix pathway.

The one or more genes of the mevalonate pathway and the one or more genes of the Nudix pathway may be encoded on one or more vectors within the host cell. For example, the polynucleotide sequences may be encoded on one vector, two vectors, three vectors, four vectors, five vectors or six vectors. It will be appreciated by a person skilled in the art that the one or more genes of the mevalonate pathway, and the one or more genes of the Nudix pathway can be located in one or more vectors in different combinations. In one example, the one or more genes of the mevalonate pathway may be encoded on one vector and the one or more genes of the Nudix pathway may be encoded on another vector. In another example, the one or more genes of the mevalonate pathway may be encoded on two vectors and the one or more genes of the Nudix pathway may be encoded on another vector. In another example, the one or more genes of the Nudix pathway may be encoded on two vectors and the one or more genes of the mevalonate pathway may be encoded on another vector. It will also be appreciated by a person skilled in the art that where there is more than one gene of a pathway, these can be encoded on separate vectors in combination with one or more genes from another pathway. It will generally be understood that the examples provided in the foregoing are not exhaustive and different combinations would be acceptable.

The one or more genes of the mevalonate pathway and the one or more genes of the Nudix pathway may in some examples be inserted into the genome of the host cell. A person skilled in the art would understand that the genomic insertion of one or more genes of the mevalonate pathway and one or more genes of the Nudix pathway into the host genome refers to the targeted and stable insertion of an exogenous gene into the host genome, allowing stable gene expression. The one or more genes of the mevalonate pathway and the one or more genes of the Nudix pathway may be inserted into the genome of the host cell using genomic modification methods including but is not limited to CRISPR-Cas9, TALEN-mediated gene knockin.

In one example, the host cell may comprise two vectors, wherein a) a first vector comprises a polynucleotide sequence encoding one or more genes of the mevalonate pathway; and b) a second vector comprises a polynucleotide sequence encoding one or more genes of the Nudix pathway.

Genes of the mevalonate pathway include but are not limited to HMG-COA synthase (hmgS), acetoacetyl-CoA thiolase (atoB), HMG-COA reductase (hmgR), mevalonate kinase (mevk), phosphomevalonate kinase (pmk), mevalonate pyrophosphate decarboxylase (pmd), (isopentenyl diphosphate) IPP isomerase (idi), isopentenyl phosphate kinase, mevalonate 3-phosphate kinase, choline kinase, and acid phosphatase. Genes of the Nudix pathway include but are not limited to NUDX1, NudI, NudA, NudB, NudC, NudH, DR2204, IaIA and MJ1149.

In some examples, the one or more genes of the mevalonate pathway are isolated from bacterium or yeast. In one example, the one or more genes of the mevalonate pathway may be isolated from a bacterium selected from the group consisting of Escherichia coli, Pantoea agglomerans, Pantoea ananatis, uncultured marine bacterium HF10_19P19, Sulfolobus solfataricus, Anabaena variabilis and Brevundimonas sp. In one example, the one or more genes of the mevalonate pathway may be isolated a yeast selected from the group consisting of Saccharomyces cerevisiae. Yarrowia lipolytica, Rhodosporidium toruloides, Candida and Pichia.

In one example, the one or more genes of the mevalonate pathway may be selected from the group consisting of HMG-COA synthase (hmgS), acetoacetyl-CoA thiolase (atoB), HMG-CoA reductase (hmgR), mevalonate kinase (mevK), phosphomevalonate kinase (pmk), mevalonate pyrophosphate decarboxylase (pmd), (isopentenyl diphosphate) IPP isomerase (idi) or combinations thereof.

In some examples, the one or more genes of the Nudix pathway are isolated from prokaryotes or plants. The prokaryote may be a bacterium or an archaea. In one example, the one or more genes of the Nudix pathway may be isolated from a bacterium selected from the group consisting of Escherichia coli, Deinococcus radiodurans, Bartonella bacilliformis. In another example, the one or more genes of the Nudix pathway may be isolated from an archaea such as Methanocaldococcus jannaschii. In another example, the one or more genes of the Nudix pathway may be isolated from a plant selected from the group consisting of Rose hybrida and Arabidopsis thaliana.

In one example, the one or more genes of the Nudix pathway may be selected from the group consisting of NUDX1, NudI, NudA, NudB, NudC, NudH, DR2204, Ia1A, MJ1149 or combinations thereof.

In one example, the polynucleotide sequence encoding atoB gene is SEQ ID NO: 19. In one example, the polynucleotide sequence encoding hmgS gene is SEQ ID NO: 20. In one example, the polynucleotide sequence encoding mevK gene is SEQ ID NO: 21. In one example, the polynucleotide sequence encoding pmk gene is SEQ ID NO: 22. In one example, the polynucleotide sequence encoding pmd gene is SEQ ID NO: 23. In one example, the polynucleotide sequence encoding idi gene is SEQ ID NO: 24. In one example, the polynucleotide sequence encoding RhNUDX1 gene is SEQ ID NO: 25 In one example, the polynucleotide sequence encoding NudI gene is SEQ ID NO: 26. In one example, the polypeptide sequence of hmgR is SEQ ID NO: 66.

The one or more genes of the mevalonate and Nudix pathway may in some examples be modified. The modification of the one or more genes may comprise mutation, truncation, translocation, substitution, deletion and insertion, or post-translation modification of the translated gene. The genes may be modified to improve the expression levels. post-translational modification of the translated protein or combinations of any of these modifications.

In one example, the hmgR gene is truncated (referred to as “thmgR”). It will be appreciated by a person skilled in the art that the term ‘truncation’ refers to elimination of the N- or C-terminal portion of a protein by manipulation of the structural gene, or premature termination of protein elongation due to the presence of a termination codon in its structural gene as a result of a nonsense mutation. In one example, the polypeptide sequence of truncated hmgR is SEQ ID NO: 27 and the polynucleotide sequence encoding the truncated hmgR gene is SEQ ID NO: 28.

In some examples, the one or more vectors may comprise a polynucleotide sequence encoding one or more diphosphate synthase genes, prenyltransferase genes, or a combination of diphosphate synthase and prenyltransferase genes. The one or more diphosphate synthase genes, prenyltransferase genes or combination of diphosphate synthase and prenyltransferase genes may be located on the first vector or the second vector or on both first and second vectors. In one example, the polynucleotide encoding the one or more diphosphate synthase genes, prenyltransferase genes or combination of diphosphate synthase and prenyltransferase genes is encoded on the second vector. It will also be appreciated by a person skilled in the art that the polynucleotide sequence on each vector may comprise a combination of diphosphate synthase genes or prenyltransferase genes. For example, the polynucleotide sequence may encode for one diphosphate synthase gene. In another example, the polynucleotide sequence may encode for one prenyltransferase gene. In another example, the polynucleotide sequence may encode for two diphosphate synthase genes. In another example, the polynucleotide sequence may encode for two prenyltransferase genes. In yet another example, the polynucleotide sequence may encode for one diphosphate synthase gene and one prenyltransferase gene. It will generally be understood that the examples provided in the foregoing are not exhaustive and different combinations would be acceptable.

The polynucleotides sequences in the one or more vectors would be understood to be operably linked to a promoter. It would generally be understood that any promoter that allows expression of the polynucleotide sequence may be employed. Examples of promoters include but are not limited to the T7 RNA polymerase promoter, the lac promoter, araBAD promoter, tac promoter, lambda cl857-PL promoter and the T5 promoter.

In some examples, the promoter may be an inducible promoter. In one example, the promoter may be naturally inducible. In one example, the promoter may be engineered to be inducible. It will be appreciated that any suitable inducible promoter system may be used. Inducible promoter systems may be induced by an inducer or stimuli including but not limited to chemical inducers, light or heat.

In one example, the polynucleotide sequence is operably linked to an inducible promoter in one or more vectors and operably linked to an uninducible promoter in the other vectors. For example, the polynucleotide sequence is operably linked to an inducible promoter in each of the vectors. In another example, the polynucleotide sequence is operably linked to an inducible promoter in two vectors and the polynucleotide sequence is operably linked to an uninducible promoter in the other vectors.

In one example, the polynucleotide sequence in each of the vectors is operably linked to an inducible promoter. In one example, the inducible promoter is a wild-type T7 RNA polymerase promoter or a variant of the wild-type T7 RNA polymerase promoter. The variant of the wild-type T7 RNA polymerase promoter may be generated via mutations to the wild-type promoter. In another example, the T7 RNA polymerase promoter variant is selected from the group consisting of TM1, TM2, TM3, TV1, TV2, TV3 and TV4. In one example, the polynucleotide sequence encoding wild-type T7 RNA polymerase promoter is SEQ ID NO: 29. In one example, the polynucleotide encoding the TM1 promoter is SEQ ID NO: 30. In one example, the polynucleotide encoding the TM2 promoter is SEQ ID NO: 31. In one example, the polynucleotide encoding the TM3 promoter is SEQ ID NO: 32. In one example, the polynucleotide sequence encoding the TV1 promoter is SEQ ID NO: 33. In one example, the polynucleotide sequence encoding the TV2 promoter is SEQ ID NO: 34. In one example, the polynucleotide sequence encoding the TV3 promoter is SEQ ID NO: 35. In one example, the polynucleotide sequence encoding the TV4 promoter is SEQ ID NO: 36.

The inducible promoter in each of the vectors may be independently selected from the wild-type T7 RNA polymerase promoter or variants. In one example, the inducible promoter in each of the vectors may be the wild-type T7 RNA polymerase promoter. In another example, the inducible promoter in each of the vectors may be the same T7 RNA polymerase promoter variant. In yet another example, the inducible promoter in each of the vectors may be different or combinations of the wild-type T7 RNA polymerase promoter and variants. It will generally be understood that apart from the examples provided herein, different combinations of inducible promoters may be used with each of the vectors of the invention.

In some examples, different combinations of inducible promoters may be used with each of the vectors to balance the genes of the mevalonate pathway, the Nudix pathway or the mevalonate and Nudix pathways and to optimize the expression level of each of the mevalonate gene or Nudix gene.

In one example, the first vector may comprise a) a polynucleotide sequence encoding the hmgS, atoB, hmgR genes of the mevalonate pathway operably linked to a first inducible promoter; and b) a polynucleotide sequence encoding the mevk, pmk, pmd and idi genes of the mevalonate pathway operably linked to a second inducible promoter.

In one example, the inducible promoter in the first vector comprising the polynucleotide sequence encoding atoB, hmgS and truncated hmgR genes of the mevalonate pathway in the host cell as described herein is TM1, the inducible promoter in the first vector comprising the polynucleotide sequence encoding mevk, pmk, pmd and idi genes of the mevalonate pathway in the host cell as described herein is TM1 and the inducible promoter in the second vector comprising the polynucleotide sequence encoding NUDX1 gene of the Nudix pathway in the host cell as described herein is TM1. In one example, the inducible promoter in the first vector comprising the polynucleotide sequence encoding atoB, hmgS and truncated hmgR genes of the mevalonate pathway in the host cell as described herein is TM1, the inducible promoter in the first vector comprising the polynucleotide sequence encoding mevk, pmk, pmd and idi genes of the mevalonate pathway in the host cell as described herein is TM2 and the inducible promoter in the second vector comprising the polynucleotide sequence encoding NUDX1 gene of the Nudix pathway in the host cell as described herein is TM1. In one example, the inducible promoter in the first vector comprising the polynucleotide sequence encoding atoB, hmgS and truncated hmgR genes of the mevalonate pathway in the host cell as described herein is TM2, the inducible promoter in the first vector comprising the polynucleotide sequence encoding mevk, pmk, pmd and idi genes of the mevalonate pathway in the host cell as described herein is TM1 and the inducible promoter in the second vector comprising the polynucleotide sequence encoding NUDX1 gene of the Nudix pathway in the host cell as described herein is TM1. In one preferred example, the inducible promoter in the first vector comprising the polynucleotide sequence encoding atoB, hmgS and truncated hmgR genes of the mevalonate pathway in the host cell as described herein is TM3, the inducible promoter in the first vector comprising the polynucleotide sequence encoding mevk, pmk, pmd and idi genes of the mevalonate pathway in the host cell as described herein is TM2 and the inducible promoter in the second vector comprising the polynucleotide sequence encoding NUDX1 gene of the Nudix pathway in the host cell as described herein is TM1. It will generally be understood that apart from the examples provided herein, different combinations of inducible promoters may be used with each of the vectors of the invention.

The one or more vectors in the host cell as described herein may further comprise one or more polynucleotide sequences encoding a ribosomal binding site (RBS). Each vector in the host cell may further comprise the polynucleotide sequence encoding the RBS or some of the vectors may further comprise the polynucleotide sequence encoding the RBS while the others do not. For example, each of the first and second vectors may further comprise the polynucleotide sequence encoding the RBS. In another example, the first vector may comprise the polynucleotide sequence encoding the RBS while the second vector does not.

It will be appreciated by one of skill in the art that the sequence encoding the RBS may be optimized for translational efficiency and the strength of the RBS with respect to the polynucleotide sequence to be translated. Optimization of a RBS would generally be understood to involve modification of the polynucleotide sequence of the RBS. The RBS may be modified by substitution, deletion, insertion or combinations thereof of one or more nucleotide bases. The RBS may be modified using degenerate oligonucleotide bases.

The polynucleotide sequence encoding the RBS may be synthesized and inserted upstream of one or more genes located in one or more vectors. For example, the RBS may be synthesized and inserted upstream of two genes in two vectors. In another example, the polynucleotide sequence encoding the RBS may be synthesized and inserted upstream of one gene in one vector. It will generally be understood that the examples provided in the foregoing are not exhaustive and different combinations would be acceptable.

In one example, the first vector may further comprise a polynucleotide sequence encoding a ribosomal binding site (RBS) upstream of the gene of the mevalonate pathway.

In some examples, the RBS in the first vector may comprise a polynucleotide sequence of SEQ ID NO: 59, SEQ ID NO: 60, SEQ ID NO: 61, SEQ ID NO: 62, SEQ ID NO: 63, SEQ ID NO: 64, SEQ ID NO: 65 or combinations thereof.

In one example, the second vector may further comprise a polynucleotide sequence encoding a ribosomal binding site (RBS) upstream of the polynucleotide sequence encoding the diphosphate synthase gene or the prenyltransferase gene.

In some examples, the RBS in the second vector may comprise a polynucleotide sequence of SEQ ID NO: 1, SEQ ID NO:2, SEQ ID NO:3, SEQ ID NO:4, SEQ ID NO: 5 or combinations thereof. In a preferred example, RBS comprises a polynucleotide sequence of SEQ ID NO: 1 or SEQ ID NO: 4.

The diphosphate synthase or the prenyltransferase in the host cell may be selected from a geranyl pyrophosphate synthase (GPPS), a farnesyl diphosphate synthase (FPPS) or a geranylgeranyl pyrophosphate synthase.

In some examples, the diphosphate synthase or the prenyltransferase in the host cell may be modified. The modification of the diphosphate synthase or the prenyltransferase may comprise mutation, truncation, translocation, substitution, deletion and insertion.

In some examples, the GPPS may be isolated from Mentha piperita, Arabidopsis thaliana, Abies grandis, Antirrhimum majus, and Clarkia breweri. In a preferred example, the GPPS is isolated from Abies grandis (AgGPPS).

In one example, the AgGPPS is truncated at the N-terminal end.

In one example, the AgGPPS is truncated between amino acid positions 2 to 85. In one example, the AgGPPS may be truncated from amino acid positions 2 to 30. In another example, the AgGPPS may be truncated from amino acid positions 2 to 50. In another example, the AgGPPS may be truncated from amino acid positions 2 to 80. In another example, the AgGPPS may be truncated from amino acid positions 2 to 84. It will generally be understood that the examples provided in the foregoing are not exhaustive and different combinations would be acceptable. In a preferred example, the AgGPPS is truncated from amino acid positions 2 to 85.

In one example, the truncated AgGPPS comprises the polypeptide sequence as set forth in SEQ ID NO: 7.

In some examples, the FPPS may be isolated from Saccharomyces cerevisiae, Escherichia coli, Neurospora crassa and Gibberella fujikuroi. In a preferred example, the FPPS is isolated from Saccharomyces cerevisiae or Escherichia coli.

In some examples, the FPPS may be modified. The modification of the FPPS may comprise mutation, truncation, translocation, substitution, deletion and insertion to improve the expression levels.

In one example, the serine residue at amino acid position 80 of the FPPS isolated from Escherichia coli is mutated to phenylalanine.

In one example, the mutated FPPS from Escherichia coli comprises the polypeptide sequence as set forth in SEQ ID NO. 9.

In one example, the FPPS is isolated from Saccharomyces cerevisiae and comprises a) a mutation of asparagine at amino acid 127 with tryptophan; or b) a mutation of phenylalanine at amino acid position 96 with tryptophan. A person skilled in the art will understand that the FPPS isolated from Saccharomyces cerevisiae comprises a combination of both mutations.

In one example, the mutated FPPS from Saccharomyces cerevisiae comprises the polypeptide sequence as set forth in SEQ ID NO. 11, SEQ ID NO. 12 or SEQ ID NO. 13

The one or more genes of the Nudix pathway may be isolated from a eukaryote. In one example, the NUDX1 of the Nudix pathway may be isolated from a plant. The plant may be but is not limited to Rosa hybrida and Arabidopsis thaliana.

In one example, the NUDX1 is isolated from Rosa hybrida and has about 70%, 75%, 80%, 85%, 90%, 95% or 100% identity with the polypeptide sequence set forth in SEQ ID NO: 14.

In another example, the NUDX1 is isolated from Arabidopsis thaliana and has about 70%, 75%, 80%, 85%, 90%, 95% or 100% identity with the polypeptide sequence set forth in SEQ ID NO: 15.

The one or more genes of the Nudix pathway may also be isolated from a prokaryote. In one example, the NudI, NudA, NudB, NudC or NudH may be isolated from a prokaryote. The prokaryote may be but is not limited to Escherichia coli, Salmonella typhi, Salmonella paratyphi B and Cedecea neteri.

In some examples, the one or more genes of the Nudix pathway may be co-expressed with the diphosphate synthase or prenyltransferase gene. In one example, the one or more genes of the Nudix pathway may be fused with the diphosphate synthase or prenyltransferase gene. It will generally be understood by a person skilled in the art that the fusion may result from structural rearrangements like translocations and deletions, transcription read-through of neighboring genes, or the trans- and cis-splicing of pre-mRNAs. It will generally be understood that the examples provided in the foregoing are not exhaustive and genetic fusion methods would be acceptable.

In some examples, the host cell may further comprise a polynucleotide sequence encoding a geranyl synthase enzyme (GES) isolated from a plant. The polynucleotide sequence encoding a geranyl synthase enzyme (GES) isolated from a plant may be located on the first or second vector. In one example, the polynucleotide sequence encoding a geranyl synthase enzyme (GES) isolated from a plant is located on the first vector. In a preferred example, the polynucleotide sequence encoding a geranyl synthase enzyme (GES) isolated from a plant is located on the second vector.

The plant may be but is not limited to Ocimum basilicum, Valeriana officinalis, Phyla dulcis, Cinnamomum tenuipile and Camptotheca acuminate.

In one example, the GES is isolated from Ocimum basilicum and comprises the polynucleotide sequence as set forth in SEQ ID NO: 16.

The polynucleotide sequence encoding the GES may be located upstream or downstream of the polynucleotide sequence encoding the diphosphate synthase or prenyltransferase. The polynucleotide sequence encoding the GES may be located upstream or downstream of the polynucleotide sequence encoding the gene of the Nudix pathway

The GES may be co-expressed with the diphosphate synthase or prenyltransferase gene, or the gene of the Nudix pathway. The GES may be co-expressed with both the diphosphate synthase gene and gene of the Nudix pathway too. In one example, the GES may be fused with the diphosphate synthase or prenyltransferase gene. It will generally be understood by a person skilled in the art that the fusion may result from structural rearrangements like translocations and deletions, transcription read-through of neighboring genes, or the trans- and cis-splicing of pre-mRNAs. It will generally be understood that the examples provided in the foregoing are not exhaustive and genetic fusion methods would be acceptable.

In some examples, the host cell may further comprise a polynucleotide sequence encoding a multiple antibiotic resistance protein (MarA). The polynucleotide sequence encoding the MarA may be located on the first vector or second vector, or incorporated into the genome of the host cell. In one example, the polynucleotide sequence encoding the MarA is located on the first vector. In a preferred example, the polynucleotide sequence encoding the MarA is located on the second vector.

The polynucleotide sequence encoding the MarA may be located upstream or downstream of the polynucleotide sequence encoding the diphosphate synthase or prenyltransferase. The polynucleotide sequence encoding the MarA may be located upstream or downstream of the polynucleotide sequence encoding the gene of the Nudix pathway.

In some examples, the host cell may further comprise a polynucleotide sequence encoding an alcohol acyltransferase (AAT) enzyme. The polynucleotide sequence encoding an alcohol acyltransferase (AAT) enzyme may be located on the first or second vector. In one example, the polynucleotide sequence encoding an alcohol acyltransferase (AAT) enzyme is located on the second vector.

The polynucleotide sequence encoding AAT may be located upstream or downstream of the polynucleotide sequence encoding the diphosphate synthase or prenyltransferase. The polynucleotide sequence encoding AAT may be located upstream or downstream of the polynucleotide sequence encoding the gene of the Nudix pathway.

In one example, the AAT enzyme is isolated from a plant. The plant may be but is not limited to Rosa hybrida.

The host cell of the present invention may be deficient in one or more genes. In some examples, the host cell may be deficient in the pta gene, ackA gene or both pta and ackA genes.

In other examples, the host cell may be deficient in at least one gene involved in amino acid synthesis, oxidation of terpenoids and amino acid degradation. It will be appreciated by a person skilled in the art that where the host cell is deficient in at least one gene, these can be a combination of genes involved in different processes. For example, the host cell may be deficient in one or more genes involved in amino acid synthesis, and one or more genes involved in oxidation of terpenoids. In another example, the host cell may be deficient in one or genes involved in amino acid synthesis, and one or more genes involved in amino acid degradation. In another example, the host cell may be deficient in one or more genes involved in oxidation of terpenoids, and one or more genes involved in amino acid degradation. It will generally be understood that the examples provided in the foregoing are not exhaustive and different combinations would be acceptable.

The gene involved in the oxidation of terpenoids may be but is not limited to yjgB, yahK and yddN. It will be appreciated by a person skilled in the art that the host cell may be deficient in a combination of genes involved in the oxidation of terpenoids.

The gene involved in amino acid synthesis may be but is not limited to aroA, aroB and serC. It will be appreciated by a person skilled in the art that the host cell may be deficient in a combination of genes involved in amino acid synthesis.

The gene involved in amino acid degradation may be but is not limited to tnaA.

It will generally be understood that the host cell may be deficient in one or more of the ack gene, pta gene, genes of the amino acid synthesis, oxidation of terpenoids and/or amino acid degradation in various combinations. In one example, the host cell is deficient in aroA, serC, yjgB, tnaA, and ack. In another example the host cell is deficient in aroA, serC, tnaA and pta genes. In a preferred example, the host cell is deficient in aroA, serC, yjgB and tnaA genes. In another preferred example, the host cell is deficient in aroA, serC and tnaA genes

The host cell may be modified to be deficient in ack gene, pta gene, genes of the amino acid synthesis, oxidation of terpenoids and amino acid degradation by genomic modification methods. Reduction in gene expression levels may be carried out using genomic modification methods including but is not limited to siRNA knockdown and shRNA knockdown. The genes may be deleted from the genome of the host cell using genomic modification methods including but is not limited to CRISPR-Cas9, FRT gene deletion, TALEN-mediated gene knockout.

As described herein, the host cell of the present invention may be a bacterial cell. The bacterial cell may be but is not limited to Escherichia, Pantoea, Bacillus, Corynebacterium, Paracoccus, Streptomyces and Synechococcus. In a preferred example, the bacterial cell is an Escherichia coli cell. It will generally be understood that any industrial bacterium or bacterial cell may be used in the present invention.

The strain of the Escherichia coli cell may be but is not limited to BL21 DE3 strain, K-12 (RV308), K-12 (HMS174), K-12 substr. MG1655, W strain (ATCC 9637), JM109 (DE3), BW25113, JM109 DE3, Mach1 and any strain comprising T7 RNA polymerase gene. In a preferred example, the Escherichia coli cell is a BL21 DE3 strain.

In another aspect, the present invention refers to an engineered fusion protein produced comprising a diphosphate synthase or prenyltransferase of the mevalonate pathway and a nudix hydrolase as described herein, a diphosphate synthase or prenyltransferase of the mevalonate pathway, a nudix hydrolase and a geranyl synthase enzyme (GES) of the terpene synthase pathway as described herein, or a diphosphate synthase or prenyltransferase of the mevalonate pathway and a GES of the terpene synthase pathway as described herein.

The polynucleotide sequence encoding the GES of the terpene synthase pathway may be located upstream or downstream of the polynucleotide sequence encoding the diphosphate synthase or prenyltransferase. The polynucleotide sequence encoding the GES of the terpene synthase pathway may be located upstream or downstream of the polynucleotide sequence encoding the gene of the Nudix pathway

In one example, the GES of the terpene synthase pathway is located between the diphosphate synthase or prenyltransferase and the gene of the Nudix pathway.

The GES of the terpene synthase pathway may be fused with the diphosphate synthase gene, prenyltransferase gene or the gene of the Nudix pathway. The GES of the terpene synthase pathway may be fused with both the diphosphate synthase gene or prenyltransferase gene, and the gene of the Nudix pathway too. It will generally be understood by a person skilled in the art that the fusion may result from structural rearrangements like translocations and deletions, transcription read-through of neighboring genes, or the trans- and cis-splicing of pre-mRNAs. It will generally be understood that the examples provided in the foregoing are not exhaustive and genetic fusion methods would be acceptable.

The diphosphate synthase or prenyltransferase of the engineered fusion protein may be upstream or downstream of the nudix hydrolase. It will generally be understood by a person skilled in the art each strand of DNA or RNA has a 5′ end and a 3′ end, named based on the carbon position on the deoxyribose (or ribose) ring. A person skilled in the art will also understand that the terms “upstream” and “downstream” refer to the orientation that reflects the direction of the synthesis of mRNA and its translation from the 5′ end to the 3′ end. The term “upstream” refers to the 5′ end of the coding strand for the gene in question and the region of the coding strand towards the 3′ end is referred to as the downstream.

The engineered fusion protein may further comprise one or more linker sequences.

In some examples, the linker sequence of the engineered fusion protein may be located between the nudix hydrolase and the diphosphate synthase or prenyltransferase of the fusion protein, wherein the linker is linked to the C-terminal of the nudix hydrolase and the N-terminal of the diphosphate synthase or prenyltransferase. In another example, the linker is linked to the N-terminal of the nudix hydrolase and the C-terminal of the diphosphate synthase or prenyltransferase.

In some examples, the linker sequence of the engineered fusion protein may comprise at least 70%, 75%, 80%, 85%, 90%, 95% and 100% sequence identity with SEQ ID NO: 17 or SEQ ID NO: 18.

In another aspect, the present invention refers to a method of geraniol, geranyl acetate, or geraniol and geranyl acetate production comprising culturing the host cell as described herein in a culture medium. The host cell may be cultured in a suitable culture vessel including but not limited to a tube, a flask or a bioreactor.

The method of geraniol, geranyl acetate, or geraniol and geranyl acetate production may further comprise the step of isolating geraniol, geranyl acetate, or geraniol and geranyl acetate from the culture medium.

The method comprises the culturing of the host cell as described herein in a culture medium. The culture medium may comprise but not limited to components in the TB medium and the 2×PY medium. Additional components may be added to the culture medium and include antibiotics, inducers and carbon substrates.

The antibiotics may be supplemented in the culture medium at the beginning of the culturing process. The antibiotics may be added continuously throughout the culturing process. The antibiotics include kanamycin and spectinomycin.

The culture medium may be further supplemented by one or more inducers capable of inducing the inducible promoter. The inducer may be added in the culture medium at the beginning of the of the culturing process. The culture medium may be supplemented with the inducer when the host cell has grown to an optical density. The culture medium may be supplemented continuously to the culture medium throughout the culturing process. In another example, the host cell may be cultured in conditions suitable for inducing the inducible promoter.

Examples of inducers include but are not limited to galactose, lactose or isopropyl β-D-1 thiogalactopyranoside (IPTG). In a preferred example, the inducer is lactose or IPTG.

The concentration of IPTG may be about 0.01 mM to about 0.15 mM. For example, the concentration of IPTG may be about 0.01 mM, about 0.02 mM, about 0.03 mM, about 0.04 mM, about 0.05 mM, about 0.06 mM, about 0.07 mM, about 0.08 mM, about 0.09 mM, about 0.10 mM, about 0.11 mM, about 0.12 mM, about 0.13 mM, about 0.14 mM and about 0.15 mM. In a preferred example, the concentration of IPTG is about 0.05 mM.

The culture medium may also comprise at least one carbon substrate which may be but is not limited to glucose, glycerol, lactose and sucrose. A person skilled in the art will understand that the culture medium may contain a single type of carbon substrate or combinations of carbon substrates. In a preferred example, the culture medium comprises lactose, glucose and glycerol.

The concentration of lactose may be about 5 mM to about 50 mM. For example, the concentration of lactose may be about 5 mM, about 10 mM, about 15 mM, about 20 mM, about 25 mM, about 30 mM, about 35 mM, about 40 mM, about 45 mM and about 50 mM. In a preferred example, the concentration of lactose is about 40 mM.

The concentration of glucose may be about 2 g/L to about 3 g/L. For example, the concentration of glucose may be about 2.0 g/L, about 2.1 g/L, about 2.2 g/L, about 2.3 g/L, about 2.4 g/L, about 2.5 g/L, about 2.6 g/L, about 2.7 g/L, about 2.8 g/L, about 2.9 g/L and about 3.0 g/L.

The concentration of glycerol may be about 8 g/L to about 30 g/L. For example, the concentration of glycerol may be about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L and about 30 g/L.

In some examples, the culture medium may comprise a nitrogen supplement. The nitrogen supplement may be but is not limited to tryptone, nitrates, ammonium, urea and proteose-peptone.

In a preferred example, the nitrogen supplement is tryptone. The concentration of tryptone may be about 1 g/L to about 10 g/L. For example, the concentration of tryptone may be about 1 g/L, about 2 g/L, about 3 g/L, about 4 g/L, about 5 g/L, about 6 g/L, about 7 g/L, about 8 g/L, about 9 g/L and about 10 g/L.

In some examples, the culture medium may comprise an organic solvent. The organic solvent may be but is not limited to dodecane, plant oils, undecane, isoamyl laurate and isopropyl myristate.

The ratio of dodecane to media may be about 0.2 to about 1.0. For example, the ratio of dodecane to media may be about 0.2, about 0.3, about 0.4, about 0.5, about 0.6, about 0.7, about 0.8, about 0.9 and about 1.0.

The culture medium may be maintained at a pH of about 6.5 to about 7.5. For example, the pH may be about 6.5, about 6.6, about 6.7, about 6.8, about 6.9, about 7.0, about 7.1, about 7.2, about 7.3, about 7.4 and about 7.5. In a preferred example, the pH is about 7.0.

The method of geraniol, geranyl acetate, or geraniol and geranyl acetate production in some examples, comprise culturing the host cell in the culture medium for about 1 day, about 2 days, about 3 days, about 4 days, about 5 days, about 6 days or about 7 days. In a preferred embodiment, the host cell is cultured in the culture medium for about 3 days or about 4 days.

In one example, after 3 days of cultivation, the yield of geraniol production in a tube may be between about 292 to about 684 mg/L. For example, the yield of geraniol production in a tube may be about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L and about 700 mg/L.

In one example, after 3 days of cultivation, the yield of geraniol production in a flask may be up to about 907 mg/L. For example, the yield of geraniol production in a flask may be about 50 mg/L, about 100 mg/L, about 150 mg/L, about 200 mg/L, about 250 mg/L, about 300 mg/L, about 350 mg/L, about 400 mg/L, about 450 mg/L, about 500 mg/L, about 550 mg/L, about 600 mg/L, about 650 mg/L, about 700 mg/L, about 750 mg/L, about 800 mg/L, about 850 mg/L and about 900 mg/L.

In one example, the carbon yield may be at least 24%, at least 30%, at least 35%, at least 40%, at least 45%, at least 50%, at least 55%, at least 60%, at least 65%, at least 70%, at least 75%, at least 80%, at least 85%, at least 90%, at least 95%, and wherein the carbon yield is calculated as a ratio of product obtained and total metabolizable carbon sources used.

The host cell may be cultured in a batch fermentation culture medium or a fed-batch fermentation culture medium.

The batch fermentation culture medium or a fed-batch fermentation culture medium may comprise but not limited to components in the TB medium and the 2×PY medium. The components that may be added to the culture medium include one or more antibiotics, one or more inducers, one or more carbon substrates and/or one or more organic solvents.

The antibiotics may be supplemented in the culture medium at the beginning of the culturing process. The antibiotics may be added continuously throughout the culturing process. The antibiotics include kanamycin and spectinomycin.

The inducer in the culture medium capable of inducing the inducible promoter may be but is not limited to galactose, lactose or isopropyl β-D-1 thiogalactopyranoside (IPTG). In a preferred example, the inducer is lactose or IPTG.

The at least one carbon substrate may be but is not limited to glucose, glycerol, lactose and sucrose. In some examples, a person skilled in the art will understand that the culture medium may contain a combination of carbon substrates.

The organic solvent in the culture medium may be but is not limited to dodecane, plant oils, undecane, isoamyl laurate and isopropyl myristate.

In some examples, the culture medium may comprise a nitrogen supplement. The nitrogen supplement may be but is not limited to tryptone, nitrates, ammonium, urea and proteose-peptone.

In one example, the batch fermentation culture medium may comprise glucose, glycerol, lactose and IPTG.

The concentration of glucose in the batch fermentation culture medium may be about 1 to about 10 g/L. For example, the concentration of glucose may be about 1 g/L, about 2 g/L, about 3 g/L, about 4 g/L, about 5 g/L, about 6 g/L, about 7 g/L, about 8 g/L, about 9 g/L and about 10 g/L. In a preferred example, the concentration of glucose in the batch fermentation culture medium is about 1 to about 2 g/L

The concentration of glycerol in the batch fermentation culture medium may be about 1 to about 30 g/L. For example, the concentration of glycerol may be about 1 g/L, about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L and about 30 g/L In a preferred example, the concentration of glycerol in the batch fermentation culture medium is about 8 to about 10 g/L.

The concentration of lactose in the batch fermentation culture medium may be about 5 mM to about 50 mM. For example, the concentration of lactose may be about 5 mM, about 10 mM, about 15 mM, about 20 mM, about 25 mM, about 30 mM, about 35 mM, about 40 mM, about 45 mM and about 50 mM. In a preferred example, the concentration of lactose is about 40 mM.

The concentration of IPTG in the batch fermentation culture medium may be about 0.01 mM to about 0.2 mM. For example, the concentration of IPTG may be about 0.01 mM, about 0.02 mM, about 0.03 mM, about 0.04 mM, about 0.05 mM, about 0.06 mM, about 0.07 mM, about 0.08 mM, about 0.09 mM, about 0.10 mM, about 0.11 mM, about 0.12 mM, about 0.13 mM, about 0.14 mM, about 0.15 mM, about 0.16 mM, about 0.17 mM, about 0.18 mM, about 0.19 mM and about 0.20 mM. In a preferred example, the concentration of IPTG is about 0.10 mM.

The batch fermentation culture medium may be supplemented with carbon substrates and IPTG when the host cell has grown to an optical density (OD₆₀₀) of about 0.5 to about 2. The optical density may be about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, and about 2.0. In a preferred example, the OD₆₀₀ is about 1.0.

In one example, the fed-batch fermentation culture medium may comprise IPTG, magnesium sulphate and glucose or glycerol.

The concentration of IPTG in the fed-batch fermentation culture medium may be about 0.01 mM to about 0.2 mM. For example, the concentration of IPTG may be about 0.01 mM, about 0.02 mM, about 0.03 mM, about 0.04 mM, about 0.05 mM, about 0.06 mM, about 0.07 mM, about 0.08 mM, about 0.09 mM, about 0.10 mM, about 0.11 mM, about 0.12 mM, about 0.13 mM, about 0.14 mM, about 0.15 mM, about 0.16 mM, about 0.17 mM, about 0.18 mM, about 0.19 mM and about 0.20 mM. In a preferred example, the concentration of IPTG is about 0.1 mM.

The concentration of glucose or glycerol in the fed-batch fermentation culture medium may be about 200 to about 750 g/L. For example, the concentration of glucose or glycerol may be about 200 g/L, about 225 g/L, about 250 g/L, about 275 g/L, about 300 g/L, about 325 g/L, about 350 g/L, about 375 g/L, about 400 g/L, about 425 g/L, about 450 g/L, about 475 g/L, about 500 g/L, about 525 g/L, about 550 g/L, about 575 g/L, about 600 g/L, about 625 g/L, about 650 g/L, about 675 g/L, about 700 g/L, about 725 g/L and about 750 g/L. In a preferred example, the concentration of glucose or glycerol in the fed-batch fermentation culture medium may be about 500 g/L.

The concentration of magnesium sulphate in the fed-batch fermentation culture medium may be about 1 to about 10 g/L. For example, the concentration of magnesium sulphate may be about 1 g/L, about 2 g/L, about 3 g/L, about 4 g/L, about 5 g/L, about 6 g/L, about 7 g/L, about 8 g/L, about 9 g/L and about 10 g/L. In a preferred example, the concentration of magnesium sulphate in the fed-batch fermentation culture medium may be about 5 g/L.

The fed-batch fermentation culture medium may be supplemented with the carbon substrates and magnesium sulphate continuously throughout the process at a feeding rate of between about 0.6 to about 6 g/L/h/reactor volume. For example, the feeding rate may be about 0.6 g/L/h/reactor volume, about 1.0 g/L/h/reactor volume, about 1.5 g/L/h/reactor volume, about 2.0 g/L/h/reactor volume, about 2.5 g/L/h/reactor volume, about 3.0 g/L/h/reactor volume, about 3.5 g/L/h/reactor volume, about 4.0 g/L/h/reactor volume, about 4.5 g/L/h/reactor volume, about 5.0 g/L/h/reactor volume, about 5.5 g/L/h/reactor volume and about 6.0 g/L/h/reactor volume.

The fed-batch fermentation culture medium may be further supplemented with IPTG when the host cell has grown to an optical density (OD₆₀₀) of about 10 to about 60. The optical density may be about 10, about 15, about 20, about 25, about 30, about 35, about 40, about 45, about 50, about 55 and about 60. In a preferred example, the OD₆₀₀ is about 30 to about 50.

The batch fermentation culture medium or a fed-batch fermentation culture medium may be maintained at a pH of about 6.5 to about 7.5. For example, the pH may be about 6.5, about 6.6, about 6.7, about 6.8, about 6.9, about 7.0, about 7.1, about 7.2, about 7.3, about 7.4 and about 7.5. In a preferred example, the pH is about 7.0.

In one example, the yield of geraniol production in the fed-batch fermentation may be at least 1 g/L. For example, the yield of geraniol production in the fed-batch fermentation may be at least 1 g/L, at least 5 g/L, at least 10 g/L, at least 15 g/L, at least 20 g/L, at least 25 g/L, at least 30 g/L, at least 35 g/L, at least 40 g/L, at least 45 g/L and at least 50 g/L.

In one example, the yield of geranyl acetate production in the fed-batch fermentation may be about 4 g/L to about 40 g/L. For example, the yield of geranyl acetate production in the fed-batch fermentation is about 4 g/L, about 5 g/L, about 10 g/L, about 15 g/L, about 20 g/L, about 25 g/L, about 30 g/L, about 35 g/L and about 40 g/L. [

In another aspect, the present invention refers to a kit producing geraniol, geranyl acetate, or geraniol and geranyl acetate, wherein the kit comprises the host cell as described herein with instructions for use.

In some examples, the host cell in the kit may be dissolved in solution or lyophilized.

In some examples, the host cell may be preserved by deep freezing.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

### Materials & Methods

### Strain and Plasmid Construction

E. coli BL21 DE3 strain was used for monoterpenoid production. Plasmids were constructed by combining the operons hmgS-atoB-hmgR and mevK-pmk-pmd-idi into the same p15A-spec (L2-8) vector with three different promoters (TM1, TM2 and TM3). The new plasmid set includes 9 plasmids, spk001-spk009 (Table 1). The plasmid carrying RhNUDX1 and various GPPS (AgGPPS, ispA_S80F and ERG20 mutants) was cloned into p15A-kan vector (as spk001-002d, Table 1). The best geraniol strain carried two plasmids sps004 and spk002 (RhNUDX1 and AgGPPS). For geranyl acetate plasmid, the gene RhAAT1 was inserted into spk002 after AgGPPS, and the resulting plasmid was named spk003. The genes tnaA, YjgB and ackA-pta were deleted with the CRISPR-Cas9 method as previously described using the gRNA listed in Table 2. The plasmids, oligos and strains used in this study were summarized in Tables 1, 2 and 3, respectively.

TABLE 1
Plasmid information

| | | Promoters of | | | |
|---|---|---|---|---|---|
| Serial | | modules² | | | |
| # | Plasmids¹ | M1⁴ | M2⁵ | M3⁶ | Remarks³ |
| 1 | sps001 | TM1 | TM1 | | |
| 2 | sps002 | TM1 | TM2 | | |
| 3 | sps003 | TM1 | TM3 | | |
| 4 | sps004 | TM2 | TM1 | | |
| 5 | sps005 | TM2 | TM2 | | |
| 6 | sps006 | TM2 | TM3 | | |
| 7 | sps007 | TM3 | TM1 | | |
| 8 | sps008 | TM3 | TM2 | | |
| 9 | sps009 | TM3 | TM3 | | |
| 10 | spk01 | | | TM1 | Only Nudx1 is in the M3 |
| | | | | | module |
| 11 | spk02 | | | TM1 | RBS4-GPPS1 |
| 12 | spk02_{—} | | | TM1 | RBS5-GPPS1 |
| | aroA2 | | | | |
| 13 | spk02_{—} | | | TM1 | RBS1-GPPS2 |
| | tGPPS | | | | |
| | spk02_{—} | | | TM1 | RBS2-GPPS2 |
| | tGPPS2 | | | | |
| 14 | spk02_s1 | | | TM1 | small linkers, fusion of |
| | | | | | NUDX1 and GPPS1 |
| 15 | spk02_s2 | | | TM1 | small linkers, fusion of |
| | | | | | NUDX1 and GPPS1 |
| 16 | spk02_m1 | | | TM1 | medium linkers, fusion of |
| | | | | | NUDX1 and GPPS1 |
| 17 | spk02_m2 | | | TM1 | medium linkers, fusion of |
| | | | | | NUDX1 and GPPS1 |
| 18 | spk3 | | | TM1 | GPPS3 |
| 19 | spk3a | | | TM1 | RBS3-GPPS3 |
| 20 | spk3b | | | TM1 | GPPS3a |
| 21 | spk3c | | | TM1 | GPPS3b |
| 22 | spk3d | | | TM1 | GPPS3c |
| 23 | spk04 | | | TM1 | spk02 + marA |
| 24 | spk04a | | | TM1 | spk02_tGPPS + marA |
| 25 | spk05 | | | TM1 | nudI and GPPS1 |
| 26 | spk06 | | | TM1 | ObGES and GPPS1 |
| 27 | spk06a | | | TM1 | cmR-ObGES and GPPS1 |
| 28 | spk06b | | | TM1 | cmR-ObGES, GPPS1 and |
| | | | | | GPPS2 |
| 29 | spk08 | | | TM1 | NUDX1, ObGES, GPPS1 |
| | | | | | and GPPS2 |
| 30 | spk09 | | | TM1 | NUDX1 and GPPS2 |
| 31 | spk10 | | | TM1 | NUDX1, GPPS1 and GPPS2 |
| 32 | spk11 | | | TM1 | NUDX1, GPPS1 and |
| | | | | | RhAAT1 |
| 33 | spk12 | | | TM1 | NUDX1, GPPS2 and |
| | | | | | RhAAT1 |

¹sps001-009 expressing two modules M1 and M2.
²The relative strengths for the TM1, TM2, TM3 promoters were about 92%, 37% and 16%, respectively to that of the T7 promoter.
³GPPSs used in this study. GPPS1 - ispA_S80F from Escherichia coli; GPPS2- truncated AgGPPS from Abies grandis; GPPS3 - Erg20_N127W from Saccharomyces cerevisiae; GPPS3a - Erg20_F96W; GPPS3c - Erg20_F96W_N127W. RBS information is in Table 2.
⁴The module M1 contains the three genes—HmgS, thmgR and atoB
⁵The module M2 contains the three genes—mevK, pmk, pmd, and idi
⁶The module M3 have different design, details are shown in Remarks

TABLE 2
Oligo sequence used in this study

| Name | Sequence | |
|---|---|---|
| spk2m1- | GGTAGTGGCGGCGGTGGCAGCGGTGGCCCGGGCAGCatgcatcatcatca | SEQ ID |
| f | ccatcacgag | NO: 37 |
| spk2m1- | CACCGCCGCCACTACCACCGCCGCCGCTGCCACCGCCACCAGTCGG | SEQ ID |
| r | GAACGGGTTGAAGC | NO: 38 |
| spk2s1- | GCGGTGGCCCGGGCAGCatgcatcatcatcaccatcacgag | SEQ ID |
| f | | NO: 39 |
| spk2s1- | GCCCGGGCCACCGCTGCCACCGCCACCAGTCGGGAACGGGTTGAAG | SEQ ID |
| r | C | NO: 40 |
| yjbB | TATGCCGCAAAAGAAGCGGG | SEQ ID |
| gRNA | | NO: 41 |
| tnaA | CACGAATGCGGAACGGTTCA | SEQ ID |
| gRNA | | NO: 42 |

TABLE 3
Strain information

| Strain | | | |
|---|---|---|---|
| ID | Host | Plasmids | Products |
| #G1 | BL21 | sps005 and spk01 | geraniol |
| #G2 | BL21 | sps005 and spk02 | geraniol |
| #G3 | BL21 | spk02 and sps01 | geraniol |
| #G4 | BL21 | spk02 and sps01 | geraniol |
| #G5 | BL21 | spk02 and sps02 | geraniol |
| #G6 | BL21 | spk02 and sps02 | geraniol |
| #G7 | BL21 | spk02 and sps03 | geraniol |
| #G8 | BL21 | spk02 and sps03 | geraniol |
| #G9 | BL21 | spk02 and sps04 | geraniol |
| #G10 | BL21 | spk02 and sps04 | geraniol |
| #G11 | BL21 | spk02 and sps05 | geraniol |
| #G12 | BL21 | spk02 and sps05 | geraniol |
| #G13 | BL21 | spk02 and sps06 | geraniol |
| #G14 | BL21 | spk02 and sps06 | geraniol |
| #G15 | BL21 | spk02 and sps07 | geraniol |
| #G16 | BL21 | spk02 and sps07 | geraniol |
| #G17 | BL21 | spk02 and sps08 | geraniol |
| #G18 | BL21 | spk02 and sps09 | geraniol |
| #G19 | BL21 | spk02 and sps09 | geraniol |
| #G20 | BL21 | spk02_aroA2 and | geraniol |
| | | sps08 | |
| #G21 | BL21 | spk02_aroA2 and | geraniol |
| | | sps08 | |
| #G22 | BL21 | spk02_aroA3 and | geraniol |
| | | sps08 | |
| #G23 | BL21 | spk02_aroA3 and | geraniol |
| | | sps08 | |
| #G24 | BL21 | spk02_aroA4 and | geraniol |
| | | sps08 | |
| #G25 | BL21 | spk02_aroA4 and | geraniol |
| | | sps08 | |
| #G26 | BL21 | spk02_aroA5 and | geraniol |
| | | sps08 | |
| #G27 | BL21 | spk02_aroA5 and | geraniol |
| | | sps08 | |
| #G28 | BL21 | spk02_tGPPS and | geraniol |
| | | sps08 | |
| #G29 | BL21 | spk02_tGPPS and | geraniol |
| | | sps08 | |
| #G30 | BL21 | spk02_s1 and sps08 | geraniol |
| #G31 | BL21 | spk02_s1 and sps08 | geraniol |
| #G32 | BL21 | spk02_m1 and sps08 | geraniol |
| #G33 | BL21 | spk02_m1 and sps08 | geraniol |
| #G34 | BL21 | spk03 and sps08 | geraniol |
| #G35 | BL21 | spk03 and sps08 | geraniol |
| #G36 | BL21 | spk03a and sps08 | geraniol |
| #G37 | BL21 | spk03a and sps08 | geraniol |
| #G38 | BL21 | spk03b and sps08 | geraniol |
| #G39 | BL21 | spk03b and sps08 | geraniol |
| #G40 | BL21 | spk03c and sps08 | geraniol |
| #G41 | BL21 | spk03c and sps08 | geraniol |
| #G42 | BL21 | spk03d and sps08 | geraniol |
| #G43 | BL21 | spk03d and sps08 | geraniol |
| #G44 | BL21 | spk04 and sps08 | geraniol |
| #G45 | BL21 | spk04 and sps08 | geraniol |
| #G46 | BL21 | spk04a and sps08 | geraniol |
| #G47 | BL21 | spk04a and sps08 | geraniol |
| #G48 | BL21 | spk05 and sps08 | geraniol |
| #G49 | BL21 | spk05 and sps08 | geraniol |
| #G50 | BL21 | spk06 and sps08 | geraniol |
| #G51 | BL21 | spk06 and sps08 | geraniol |
| #G52 | BL21 | spk06a and sps08 | geraniol |
| #G53 | BL21 | spk06a and sps08 | geraniol |
| #G54 | BL21 | spk06b and sps08 | geraniol |
| #G55 | BL21 | spk06b and sps08 | geraniol |
| #G56 | BL21 | spk07 and sps08 | geraniol |
| #G57 | BL21 | spk07 and sps08 | geraniol |
| #G58 | BL21 | spk07a and sps08 | geraniol |
| #G59 | BL21 | spk07a and sps08 | geraniol |
| #G60 | BL21 | spk08 and sps08 | geraniol |
| #G61 | BL21 | spk08 and sps08 | geraniol |
| #G62 | BL21 | spk09 and sps08 | geraniol |
| #G63 | BL21 | spk09 and sps08 | geraniol |
| #G64 | BL21 | spk09a and sps08 | geraniol |
| #G65 | BL21 | spk09a and sps08 | geraniol |
| #G66 | BL21 | spk10 and sps08 | geraniol |
| #G67 | BL21 | spk10 and sps08 | geraniol |
| #G68 | BL21 | sps04 and spk09 | geraniol |
| #G69 | BL21 | sps04 and spk09 | geraniol |
| #G70 | BL21 | sps01 and spk09 | geraniol |
| #G71 | BL21 | sps01 and spk09 | geraniol |
| #G72 | BL21 ΔaroAΔserC | sps08 and spk02 | geraniol |
| #G73 | BL21 ΔaroAΔserC | sps08 and spk02 | geraniol |
| #G74 | BL21 ΔaroAΔserC | sps08 and spk02 | geraniol |
| #G75 | BL21 | sps08 and spk02 | geraniol |
| | ΔaroAΔserCΔyjgB | | |
| #G76 | BL21 | sps08 and spk02 | geraniol |
| | ΔaroAΔserCΔyjgB | | |
| #G77 | BL21 | sps08 and spk02 | geraniol |
| | ΔaroAΔserCΔyjgB | | |
| #G78 | BL21 ΔaroAΔserC | sps04 and spk09 | geraniol |
| #G79 | BL21 ΔaroAΔserC | sps04 and spk09 | geraniol |
| #G80 | BL21 ΔaroAΔserC | sps04 and spk09 | geraniol |
| #G81 | BL21 | sps04 and spk09 | geraniol |
| | ΔaroAΔserCΔyjgB | | |
| #G82 | BL21 | sps04 and spk09 | geraniol |
| | ΔaroAΔserCΔyjgB | | |
| #G83 | BL21 | sps04 and spk09 | geraniol |
| | ΔaroAΔserCΔyjgB | | |
| #G84 | BL21 ΔaroAΔserC | sps01 and spk09 | geraniol |
| #G85 | BL21 ΔaroAΔserC | sps01 and spk09 | geraniol |
| #G86 | BL21 ΔaroAΔserC | sps01 and spk09 | geraniol |
| #G87 | BL21 | sps01 and spk09 | geraniol |
| | ΔaroAΔserCΔyigB | | |
| #G88 | BL21 | sps01 and spk09 | geraniol |
| | ΔaroAΔserCΔyjgB | | |
| #G89 | BL21 | sps01 and spk09 | geraniol |
| | ΔaroAΔserCΔyjgB | | |
| #G90 | BL21 | sps08 and spk11 | geranyl |
| | | | acetate |
| #G91 | BL21 | sps08 and spk11 | geranyl |
| | | | acetate |
| #G92 | BL21 | sps08 and spk11 | geranyl |
| | | | acetate |
| #G93 | BL21 | sps01 and spk11 | geranyl |
| | | | acetate |
| #G94 | BL21 | sps01 and spk11 | geranyl |
| | | | acetate |
| #G95 | BL21 | sps01 and spk11 | geranyl |
| | | | acetate |
| #G96 | BL21 | sps04 and spk11 | geranyl |
| | | | acetate |
| #G97 | BL21 | sps04 and spk11 | geranyl |
| | | | acetate |
| #G98 | BL21 | sps04 and spk11 | geranyl |
| | | | acetate |
| #G99 | MG1655 T7 | sps08 and spk02 | geraniol |
| #G100 | BL21 ΔtnaA | sps04 and spk02 | geraniol |
| #G101 | BL21 | sps04 and spk12 | geranyl |
| | | | acetate |
| #G102 | BL21 ΔyjgBΔtnaA | sps04 and spk02 | geraniol |
| #G103 | BL21 ΔyjgBΔtnaA | sps04 and spk11 | geranyl |
| | | | acetate |

Under the CRISPR-Cas9 method, different pTarget plasmids with various sgRNAs were obtained by restriction free (RF) cloning methods. The asymmetric homology arm (HA) donor DNA was amplified from the E. coli genome using iProof PCR mix (BioRad) and column purified by Zymoclean Gel DNA Recovery Kit (Zymo Research). Generally, 100-200 ng/μl of donor DNA in 30 μl can be obtained in a 100 μl PCR reaction. For the primer design, the forward primer is a fusion of the upstream homology arm (40-45 bp) sequence and downstream homology arm (15-20 bp) sequence. The 15-20 bp downstream homology arm is for annealing during initial cycles of PCR, and its length is chosen based on Tm˜50° C. The total length of forward primer is kept at 60 bp. The reverse primer is a normal PCR primer about 15-20 bp with Tm˜50° C. The length of downstream homology arms can be varied based on the reverse primer chosen. The downstream homology arm length was kept at 500 bp. BL21 chemical competent cells were prepared using the Mix & Go! E. coli Transformation Kit (Zymo Research). For the construction of BL21 cells harbouring the pCas plasmid, 10 μl of cells were mixed with 50 ng/μl of pCas plasmid and heat shocked at 42° C. for 45 s. Te cell was rescued in 200 μl of LB broth, at 30° C., 300 rpm for 1 h before spreading onto LB agar containing kanamycin (50 μg/ml) and incubated overnight at 30° C. A single colony was picked and inoculated into 1 ml LB medium containing kanamycin (50 μg/ml) and incubated at 30° C., 300 rpm overnight for making electrocompetent cells. For the preparation of electrocompetent cells, OD₆₀₀ 0.1 of the overnight BL21 cell culture harbouring the pCas plasmid was inoculated into 10 ml of LB medium containing kanamycin (50 μg/ml) and cultured at 30° C., 300 rpm. 20 mM arabinose was added to the culture at OD₆₀₀ 0.2 for the induction of A-Red recombinase. The bacterial cells were harvested at OD₆₀₀ 0.6 and centrifuged at 3800 rpm for 10 min at 4° C. The supernatant was discarded and the cells were re-suspended in 10 ml 10% glycerol. The washing step was repeated twice. The electrocompetent cells was then suspended in 100 μl of 10% glycerol. For electroporation, 20 μl of cells were mixed with 100 ng/μl of pTarget plasmid and 100 ng of donor DNA in the 1 mm Gene Pulser cuvette (Bio-Rad) and electroporated at 1.8 kV. The cells were rescued in 500 μl of LB broth, at 30° C., 300 rpm for 3 h before spreading onto LB agar containing kanamycin (50 μg/ml) and spectinomycin (100 μg/ml) and incubated overnight at 30° C. Colonies were screened by colony PCR using 2×PCRBIO Ultra Mix (PCR Biosystems) along with an unedited BL21 strain as control. The plasmids, oligos and strains used in this study are summarized in Tables 1, 2 and 3, respectively.

### Enzyme Fusion

The RhNUDX1 was fused with ispA_S80F with the orientation of RhNUDX1-ispA_S80F. Two linkers (short, GGGGSGGPGS (SEQ ID NO: 17); and medium, GGGGSGGGGSGGGGSGGPGS (SEQ ID NO: 18) were used (Table 4). The two fusion proteins were obtained using the primers (spk2 ml-f/r and spk2s1-f/r) in Table 2 with the in-house cloning method modified from Agilent QuikChange II method. Specifically, the PCR fragments with 14 bps complementary extensions to the vector ends, are amplified using the iProof™ High-Fidelity DNA Polymerase. A gel check is done to ensure amplified product size is correct. The amplified DNA fragments undergo 3 h Dpnl treatment. Thereafter the PCR product is purified using Omega PCR Cycle Pure Kits. It is then treated with Takara infusion cloning mix for 15 mins at 50° C. 1 μL of the treated product is transformed into 20 μL of DH5a competent cells, rescued for 1 h and then plated on LB plate supplemented with 50 μg/mL of kanamycin.

TABLE 4
RBS sequence for various GPPS used in this study

| | | RBS | | Protein sequence |
|---|---|---|---|---|
| No. | GPPS | ID | RBS | (N-term 35 NT) |
| 1 | AgGPPS | RBS- | Gagatataataacgagataaggaaaaga | Atgttcgatttcaacaaatacatggacag |
| | (GPPS2) | 1 | caaa (SEQ ID NO: 43) | caaagccatga (SEQ ID NO: 44) |
| 2 | AgGPPS | RBS- | gccgagatataataacgagataaggaaaa | Atgttcgatttcaacaaatacatggacag |
| | (GPPS2) | 2 | gacaaa (SEQ ID NO: 45) | caaagccatga (SEQ ID NO: 46) |
| 3 | ERG20_ | RBS- | Tgccaactttaaaagaggcctaaa (SEQ | Atggcttcagaaaaagaaattaggaga |
| | N127W | 3 | ID NO: 47) | gagagattcttga (SEQ ID NO: 48) |
| | (GPPS3) | | | |
| 4 | ERG20_ | RBS- | gccgagatataataacgagataaggaaaa | Atggcttcagaaaaagaaattaggaga |
| | N127W | 2 | gacaaa (SEQ ID NO: 49) | gagagattcttga (SEQ ID NO: 50) |
| | (GPPS3) | | | |
| 5 | ERG20_ | RBS- | Tgccaactttaaaagaggcctaaa (SEQ | Atggcttcagaaaaagaaattaggaga |
| | 96_127 | 3 | ID NO: 51) | gagagattcttga (SEQ ID NO: 52) |
| | (GPPS3b) | | | |
| 6 | ERG20 | RBS- | gccgagatataataacgagataaggaaaa | Atggcttcagaaaaagaaattaggaga |
| | 96_127 | 2 | gacaaa (SEQ ID NO: 53) | gagagattcttga (SEQ ID NO: 54) |
| | (GPPS3b) | | | |
| 7 | IspA | RBS- | Tttgtttaactttaagaaggagatatacat | Atgcatcatcatcaccatcacgagctcga |
| | (GPPS1) | 4 | (SEQ ID NO: 55) | ctttccgcagc (SEQ ID NO: 56) |
| 8 | IspA | RBS- | Tttgtttaactttaacaaggagggatacat | Atgcatcatcatcaccatcacgagctcga |
| | (GPPS1) | 5 | (SEQ ID NO: 57) | ctttccgcagc (SEQ ID NO: 58) |

### Media and Culture Conditions

Chemically defined medium (or defined medium) contained 10 g/L glucose, 2 g/L (NH₄)₂SO₄, 4.2 g/L KH₂PO₄, 11.24 g/L K₂HPO₄, 1.7 g/L citric acid, 0.5 g/L MgSO₄ and 10 ml/l trace element solution, pH 7.0. The trace element solution (100×) contained 0.25 g/L CoCl₂·6H₂O, 1.5 g/L MnSO₄·4H₂O, 0.15 g/L CuSO₄·2H₂O, 0.3 g/L H₃BO₃, 0.25 g/L Na₂MoO₄·2H₂O, 0.8 g/L Zn(CH₃COO)₂, 5 g/L Fe(III) citrate and 0.84 g/L EDTA, pH 8.0.

Semi chemically defined medium was the same as defined medium except that 2 g/L tryptone was supplemented for the geraniol production in flasks.

Auto-induction defined medium (AIDM): 2-3 g/L glucose, 8-30 g/L glycerol and 5-50 mM lactose (as inducer). The rest components were the same as defined medium. Terrific Broth (TB, 12 g/L tryptone, 24 g/L yeast extract, 2.31 g/L KH₂PO₄, and 12.54 g/L K2HPO₄) containing 2-3 g/L glucose and 20-30 g/L glycerol was used for geranyl acetate production. For auto-induction of geranyl acetate, 5-50 mM lactose was used as inducer.

For strain and abiotic optimization, the cells were grown in 1 mL of defined or AID medium in 14 ml BD Falcon™ tube at 28° C./300 rpm for 3 days. In addition, 200 μL of dodecane was used to extract monoterpenes during cell culture. When using the defined media, cells were initially grown at 37° C./300 rpm until OD₆₀₀ reached 1-2, induced by 0.01˜0.15 mM IPTG, and were then grown at 28° C./300 rpm for 2 days. For AID media, cells were grown at 28° C./300 rpm for 3 days and automatically induced by lactose. All the cultures were supplemented with the antibiotics (50 μg/ml kanamycin and 100 μg/ml spectinomycin) to maintain the two plasmids.

Flasks conditions (100, 200 and 300 rpm).

For geraniol production, cells were inoculated in 10 mL of AIDM or semi chemically defined medium in 125 mL baffled flasks at 28° C., 100-300 rpm for 3 days. 10-100% of dodecane/sunflower oil was used as product extractant. On the other hand, TB auto-induction medium was used for geranyl acetate and was supplemented with 20% dodecane/sunflower oil. The rest of the set up were the same as geraniol.

All the cultures were supplemented with the antibiotics (50 μg/ml kanamycin and 100 μg/ml spectinomycin) to maintain the two plasmids.

### Bioreactor Fermentation

Both 500 ml Mini Bioreactors and 7 L Bioreactor (Applikon Biotechnology) were used with the working volume of 200-400 mL and 2-5 L, respectively, in this study. The cells (−80° C. stock) were grown in 10 ml defined medium for 48 h at 37° C. Two modes were tested. The first mode was auto-induced batch fermentation, in which no additional nutrients were fed but the initial media contained 10 g/L glucose, 14-20 g/L lactose, 50 g/L glycerol, 4-6 g/L of ammonium sulphate and 1.5 g/L MgSO₄, other components were kept the same as in the defined media. The second mode was fed-batch fermentation, in which the process was similarly as previously described. Briefly, once OD reached about 5-6, feed solution (500 g/L glucose and 5 g/L MgSO₄) was added into the bioreactor in an exponential manner calculated on the growth rate of 0.6 h⁻¹. The cells were induced by 0.1 mM IPTG when OD reached about 30-50 (16-18 h from inoculation). After induction, a constant feeding rate at 7.5 g/L/h of glucose and 0.075 g/L/h of MgSO₄ was maintained. The culture temperature was adjusted to 30° C. and 15-20% (v/v) of dodecane was supplemented into the bioreactor. During the fermentation, dissolved oxygen level was maintained at 30% (800-2000 r.p.m) by supplying filtered air at a gas rate of 1.5 vvm. The pH of the culture was controlled at 7.0 with 28% ammonia solution. The fed-batch experiments were performed in the defined media without any antibiotics.

### Theoretic Yield of Geraniol and Geranyl Acetate

The production of isoprene or isopentenyl pyrophosphate (IPP) via the mevalonate pathway under aerobic fermentation requires three acetyl coenzyme A (AcCoA), three ATP and two NAD(P)H. Therefore,
(1) 1.5 Glucose (or 3 glycerol)+2 O₂ à 3 AcCoA+3 ATP+3 CO₂+6 NAD(P)H (glycolysis)
(2) 3 AcCoA+2 NAD(P)H à MVA
(3) MVA+3 ATP à IPP+CO₂
(4) 2 IPP à Geraniol
(5) Geraniol+AcCoA à Geranyl acetate
   Overall, 3 Glucose (or 6 glycerol)+4 O₂ à Geraniol+8 CO₂+10 H₂O, and 7/2 Glucose (or 7 glycerol)+14/3 O₂ à geranyl acetate+9 CO₂+10 H₂O, therefore, geraniol and geranyl acetate mass yields on glucose or glycerol are 28.6% and 31.1%, respectively.

### Quantification of Terpenoids

The terpenoid samples were prepared by diluting 0.5-20 μl of organic layer into 1000 μl hexane. The samples were analyzed on an Agilent 7890 gas chromatography equipped with an Agilent 5977B MSD. Samples were injected into Agilent VF-WAXms column with a split ratio of 40:1 at 240° C. The oven program started at 100° C. for 1 min, was raised up to 150° C. at 50° C./min, then to 240° C. at 15° C./min and maintained at 240° C. for another 2 min. The compound concentrations were calculated by interpolating with a standard curve prepared by authentic terpene standards (MilliporeSigma, Singapore). As the citral standard has two peaks (α- and β-citral), their concentrations were estimated based on the relative ratio of their GC chromatogram peak areas. Mass spectrometer was operated in El mode with full scan analysis (m/z 30-300, 2 spectra/s).

### Results

### In-Vitro Characterization of RhNUDX1

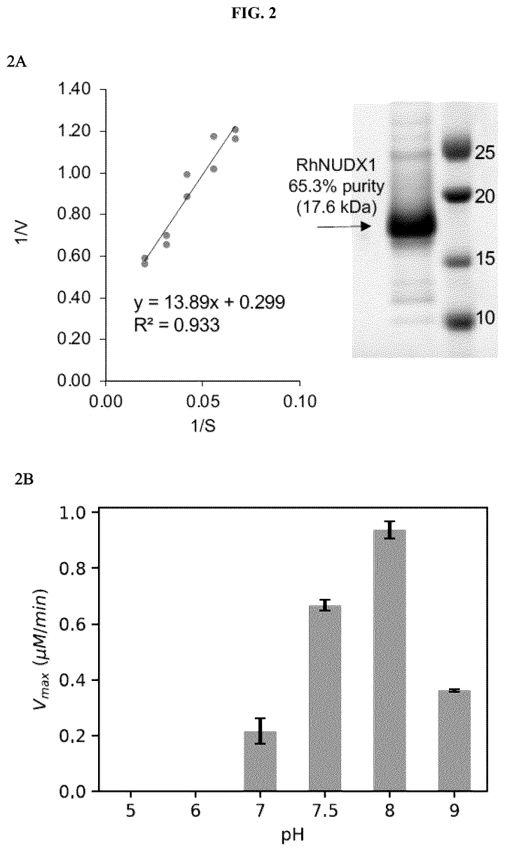

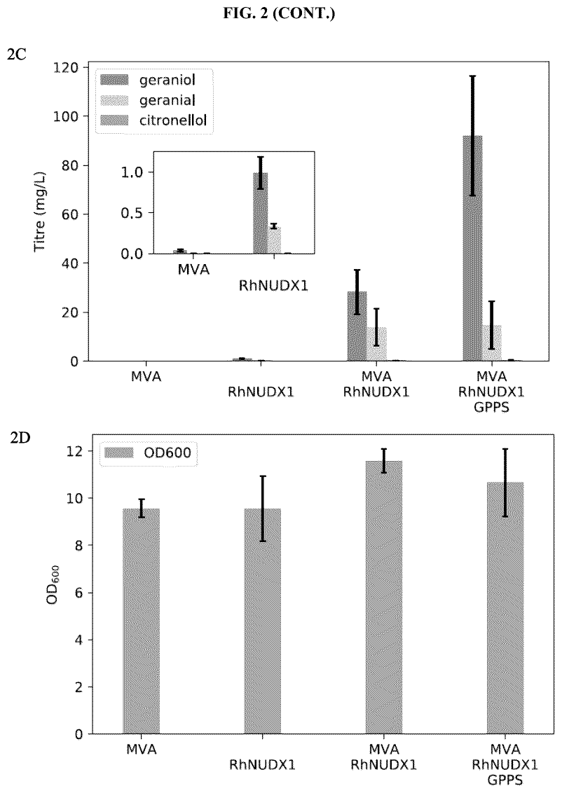

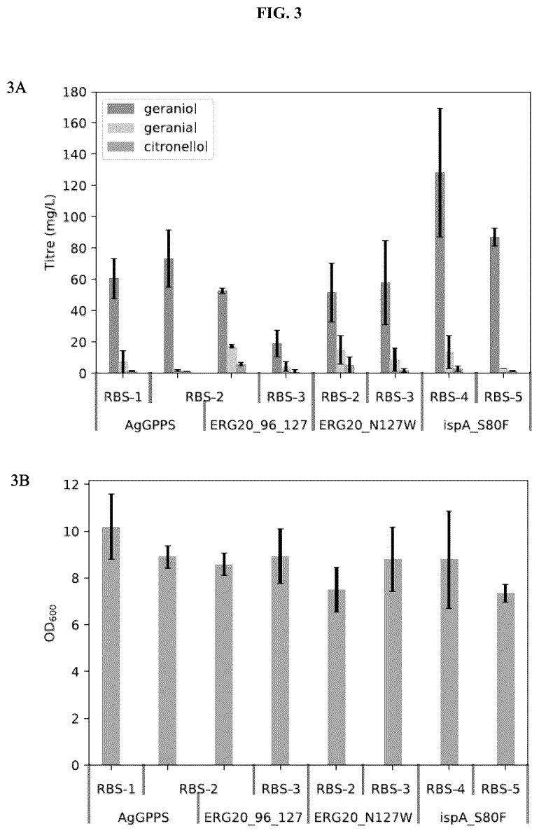

Before using RhNUDX1 for in vivo production of geraniol, RhNUDX1 was first expressed in E. coli BL21 strain and the enzyme was purified. Based on the characterization, the K_{cat} and Kₘ values of the purified RhNUDX1 (˜65.3% purity, FIG. 2A) are 0.36±0.07 s⁻¹ and 46.4±4.4 μM, respectively. Here, the k_{cat} and Kₘ values are higher than previously reported ones (Table 5), which is possibly because that NusA-RhNUDX1 fusion protein was used in previous study, while the present study used the wildtype RhNUDX1 for enzyme characterization. In both cases, the k_{cat}/Kₘ values are comparable or even better than that of the GES from Ocimum basilicum (sweet basil) (Table 5). The optimal pH, and RhNUDX1 has highest in vitro activity at pH of 8 and it completely loses activity when pH drops below 6 (FIG. 2B).

TABLE 5
Enzymes information.

| Enzymes | RhNUDX1 | NudI | ObGES | CtGES | CaGES |
|---|---|---|---|---|---|
| UniProt | JQ820249 | P52006 | AY362553 | CAD29734 | ALL56347 |
| Accession | | | | | |
| no. | | | | | |
| Enzyme | GPP | Nucleoside | Geraniol | Geraniol | Geraniol |
| name | phosphohydrolase | triphosphatase | synthase | synthase | synthase |
| | | NudI | | | |
| Organism | Rosa hybrid | Escherichia | Ocimum basilicum | Cinnamomum | Camptotheca |
| | cultivar | coli | (Sweet Basil) | tenuipile | acuminata |
| | | (strain K12) | | | (Happy |
| | | | | | tree) |
| Kingdom | Plant | Bacteria | Plant | Plant | Plant |
| Kₘ (μM) | 46.4 ± 4.4 | 310 ± 20 | 21 | 55.8 | 89.5 |
| | (0.14) | | | | |
| k_{cat} (s−1) | 0.36 ± 0.07 | 11 | 0.8 | / | / |
| | (0.02) | | | | |
| k_{cat}/Kₘ (s⁻¹ | 7747 ± 1736 | 3.6 × 10⁵ | 3.8 × 10⁴ | / | / |
| M⁻¹) | (1.4 × 10⁵) | | | | |
| Reference | This study | Protein | Plant Physiol. | Phytochemistry | J. Ind. |
| | (Science | Sci. | 134: 370- | 66: 285- | Microbiol. |
| | 349: 81- | 2019; 28(8): | 379(2004) | 293(2005) | Biotechnol. 43, |
| | 83(2015)) | 1494-1500 | | | 1281-1292 |
| | | | | | (2016) |

| GPPS | | | |
|---|---|---|---|
| enzymes | ispA | AgGPPS | ERG20 |
| UniProt | P22939 | Q8LKJ2 | P08524 |
| Accession | | | |
| no. | | | |
| Enzyme | Farnesyl diphosphate | Geranyl | Farnesyl diphosphate |
| name | Synthase | diphosphate | synthase |
| | | synthase | |
| Organism | Escherichia coli | Abies grandis | Saccharomyces |
| | (strain K12) | | cerevisiae |
| Mutations | S80F | ΔN85¹ | N127W |
| | | | F96W² |
| Functions of | Convert from a FPPS | Remove | Convert from a FPPS |
| mutations | to a GPPS | signal peptide | to a GPPS |
| Reference | Biotechnology and | Metabolic | ACS. Synth. Biol. 2014, |
| | Bioengineering 87, | Engineering | 3 (5), 298-306. |
| | 200-212 (2004). | 19 (2013) 33-41 | |

¹N-terminal truncation of the 2nd-85th amino acids
²Both single and double mutants were used: N127W, N127W-F96W

Using RhNUDX1 to Produce Geraniol in E. coli

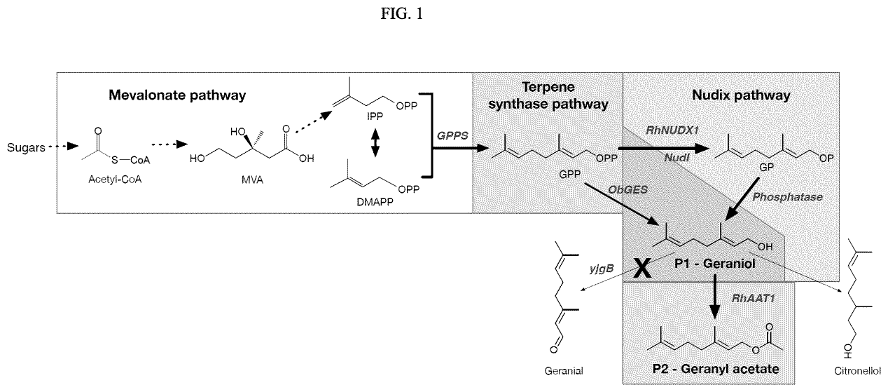

Next, RhNUDX1 was expressed in wildtype E. coli. Indeed, it produced low amount of geraniol (˜1.0 mg/L), also, 0.3 mg/L of geranial (or α-citral) was detected and trace amount of citronellol as by-products (FIG. 2C). The mevalonate pathway genes were then overexpressed (FIG. 1), to supply more terpene precursors (isopentenyl diphosphate, or IPP and dimethylallyl pyrophosphate, or DMAPP), which boosted the geraniol yield to ˜28.2 mg/L. Interestingly, only about 0.04 mg/L of geraniol in E. coli cells was detected expressing the mevalonate pathway genes but not RhNUDX1, which could be the result of native E. coli enzymes (e.g., NudB, AphA). As GPP is the intermediate of farnesyl diphosphate (FPP, C15) synthase (ispA) enzyme in E. coli and quickly converted into FPP, the truncated GPPS from Abies grandis (AgGPPS) was further expressed to increase GPP supply (FIG. 1). By combining the overexpression of GPPS and mevalonate pathway enzymes, the geraniol production was increased to 92.0 mg/L, together with about 14.8 mg/L of geranial and 0.4 mg/L of citronellol. For all the strains, cell densities were similar at the range of 9.6-11.6 (FIG. 2D), indicating that cells were not severely inhibited by the amount of geraniol produced.

### Enhancing the Supply of GPP, the Monoterpene Precursor

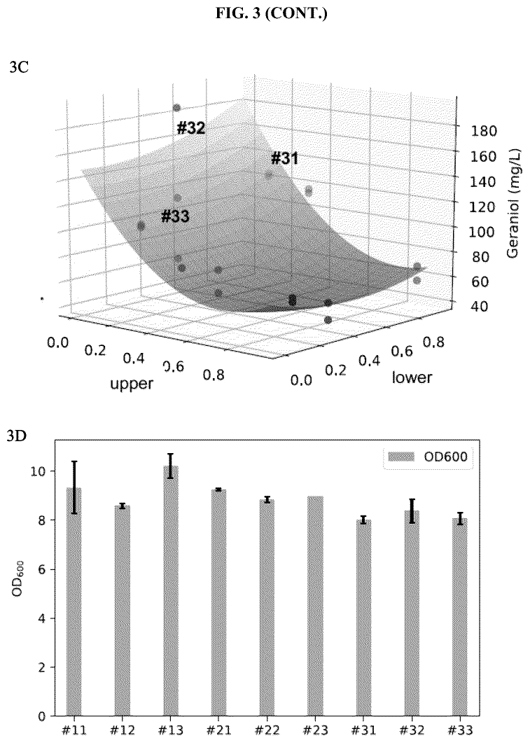

As the overexpression of GPPS resulted in over 3-fold increase in geraniol production, geraniol production might be still limited by GPP. Hence, various GPPSs were explored: AgGPPS, two mutants of FPP synthase (ERG20) from Saccharomyces cerevisiae; the mutant of FPP synthase (IspA_S80F) from E. coli. Based on previous study, the two ERG20 mutants with a reduced FPP synthase activity were selected for geraniol production: ERG20_N127W and F96W-N127W (or ERG20_96_127). In addition, the ribosomal binding sites (RBSs) of various GPPSs were perturbed (see details in Table 3). The combination of IspA_S80F and RBS-4 was found to have led to the highest production of geraniol, ˜128.2 mg/L (FIG. 3A). AgGPPS strain produced more geraniol (61-73 mg/L) than the two yeast ERG20 mutants (19-58 mg/L), but less efficient than IspA_S89F. In addition, for eukaryotic GPPSs, RBS-2 led to higher geraniol production than RBS-1 or RBS-3; for bacterial GPPS (IspA_S80F), RBS-4 was better than RBS-5 (FIG. 3A). In addition, the biomass for all the strains was similar (OD₆₀₀ values of 8-10), except that AgGPPS with RBS-1 had slightly higher OD₆₀₀ (FIG. 3B). These data indicated that the combination of RBS engineering and selection of suitable GPPSs was an effective strategy.

Pathway Balancing and Substrate Channelling by Fusing GPPS with RhNUDX1

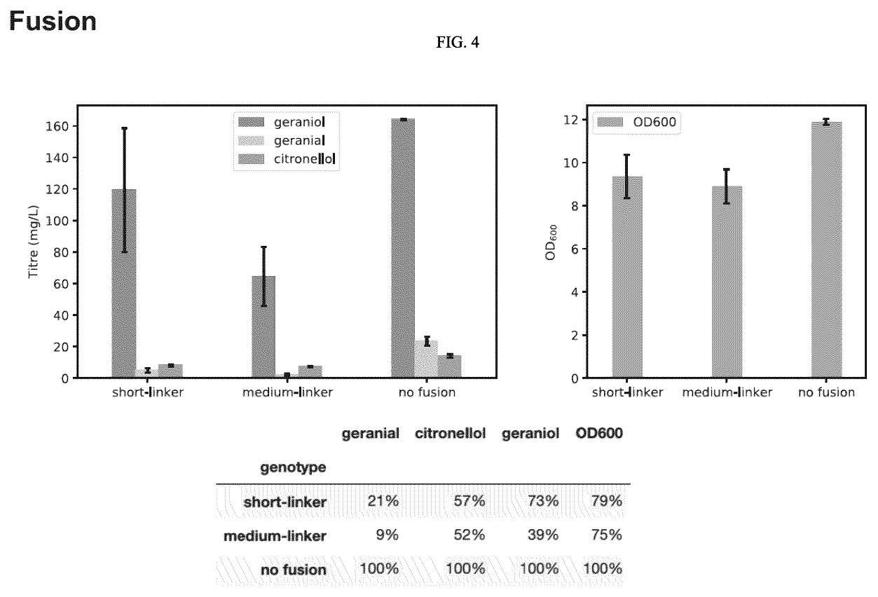

After optimized the GPP supply, the mevalonate pathway was further fined tuned transcriptionally to optimize the supply of terpene precusors (IPP and DMAPP) (FIG. 1). By dividing the mevalonate pathway into two modules (upper module: including the genes atoB, hmgS and truncated hmgR; lower module: including the genes mevk, pmK, pmd and idi), the pathway was systematically balanced by promoter engineering and identified the best (strain #32 and #31, FIGS. 3C and D). The strains #32, #31 produced about 181 and 124 mg/L of geraniol, respectively. After the mevalonate pathway balance, substrate channelling strategy was further tested. The rationale was that in E. coli, the native ispA might compete with RhNUDX1 for GPP as their substrate. The fusion of GPPS with RhNUDX1 could facilitate NUDX1 to access GPP immediately after GPP was produced by GPPS. Such strategy has been successfully applied to produce C11 terpenoids with markedly increased yields. With the orientation of RhNUDX1-GPPS, two linkers were compared: short (GGGGSGGPGS (SEQ ID NO: 17)) and medium (GGGGSGGGGGGGGSGGPGS (SEQ ID NO: 18)). The geraniol yield of short linker (119 mg/L) was about as twofold high as that of medium linker (64 mg/L). However, both designs have lower yields than the non-fused GPPS and NUDX1 (FIG. 4). This could be due to the fusion protein negatively affect the protein folding. Further study is required to understand the underlying mechanism.

### Removing Geraniol Degrading Pathways and Abiotic Optimization

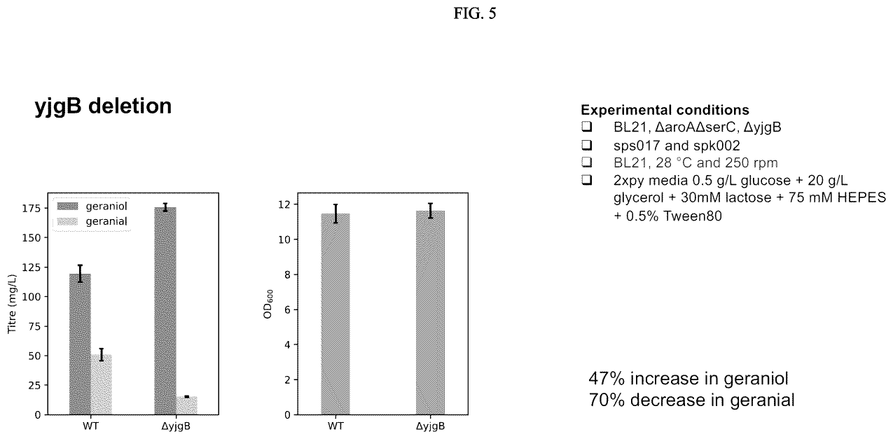

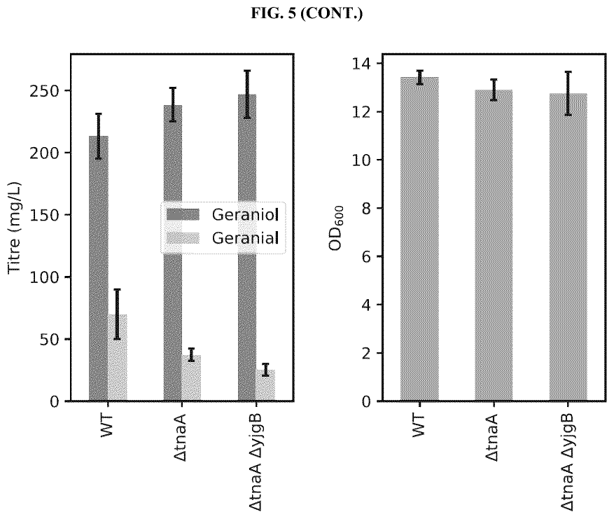

Next, the aim was to reduce the production of byproducts (geranial and citronellol). In previous constructed strains, different strains were observed to have different product distribution. Some strains produced up to 30% (the percentage was calculated by normalizing with the total yield of the three monoterpenes: geraniol, geranial and citronellol) of geranial (FIG. 2C) and some produced only 2% of geranial (AgGPPS with RBS-2, FIG. 3A). Similarly, some strain produced ˜8% and some had lower than 1% of citronellol. As geranial is the main by-product, the gene yjgB (an alcohol dehydrogenase), which was reported to be responsible for the oxidation of geraniol to geranial, was deleted. Indeed, yjbB deletion strain resulted in the increase in geraniol yield by 47% from 119 to 176 mg/L. Simultaneously, the geraniol production was reduced by 70% from 51 to 15 mg/L (FIG. 5).

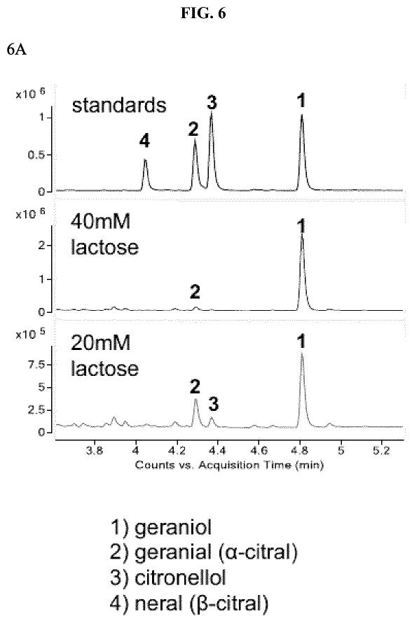

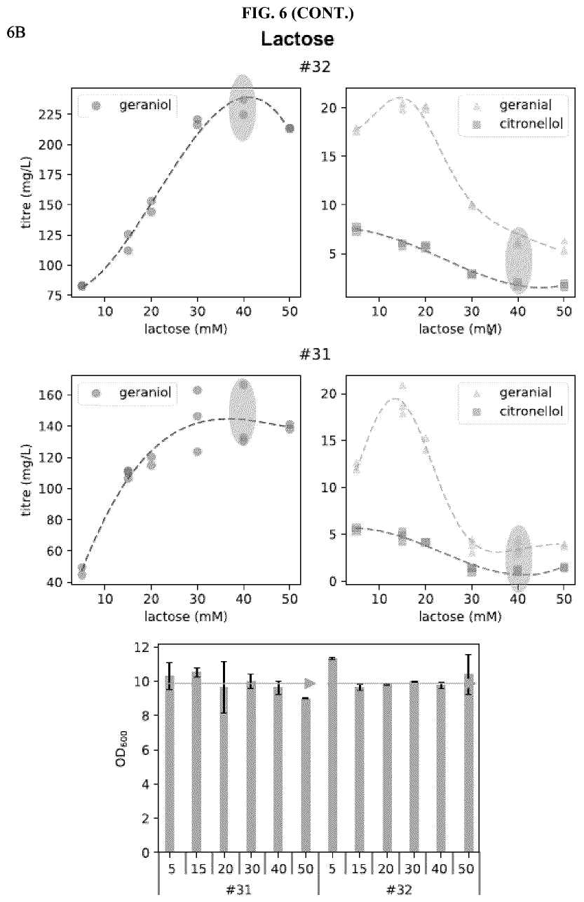

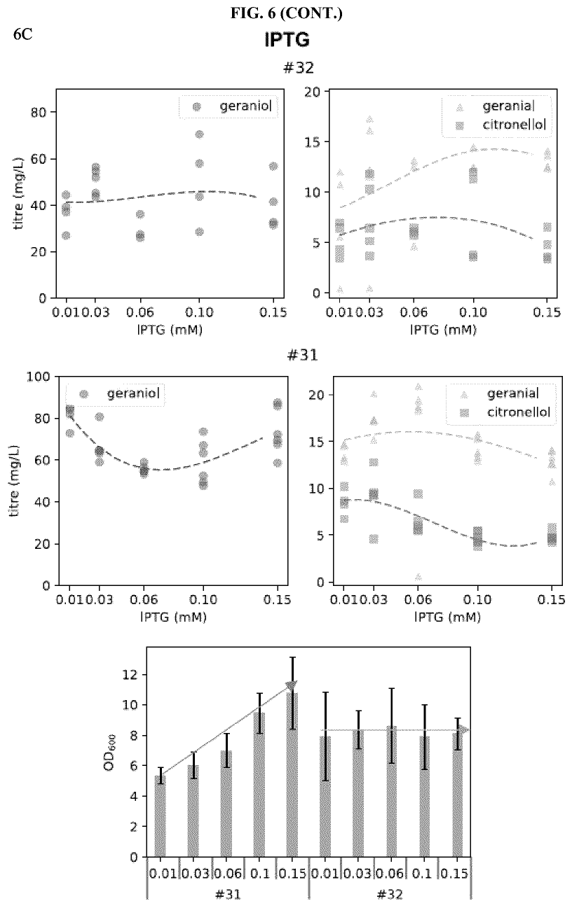

Interestingly, during these experiments, the carbon sources and inducers were also observed to have dramatic effects on the distribution. Specifically, geraniol percentage in the auto-induction defined media was higher than that in IPTG-induced defined media. In view of the serendipitous finding, the tuning of auto-induction media and inducers was further explored. With the two best strain #32 and #31, series of different concentrations of lactose or IPTG were tested. The geraniol production gradually increased as lactose concentration increases and plateaued at 40 and 30 mM of lactose for strains #32 and #31, respectively (FIGS. 6A and B). In the meantime, the production of geranial and citronellol decreased and remained lowest at 30-50 mM of lactose (For strain #32, 40-50 mM of lactose; for strain #31, 30-50 mM of lactose). In the best condition at 40 mM lactose, strain #32 produced 219, 10, 2.9 mg/L of geraniol, geranial and citronellol, respectively (i.e., the geraniol percentage was close to 97%). In contrast, the tuning of IPTG dosages had no clear effect on the production of various monoterpenes for both strains #32 and #31 (FIG. 6C). Overall, in IPTG-induced defined media, the percentage of geranial (7-18 mg/L, or 15-22%) and citronellol (4.5-9 mg/L or 5-11%) were relatively high and the geraniol production (60-80 mg/L, or 65-80%) was relatively low as compared to lactose auto-induction media.

### Exploration of Other Enzymes and Combinations

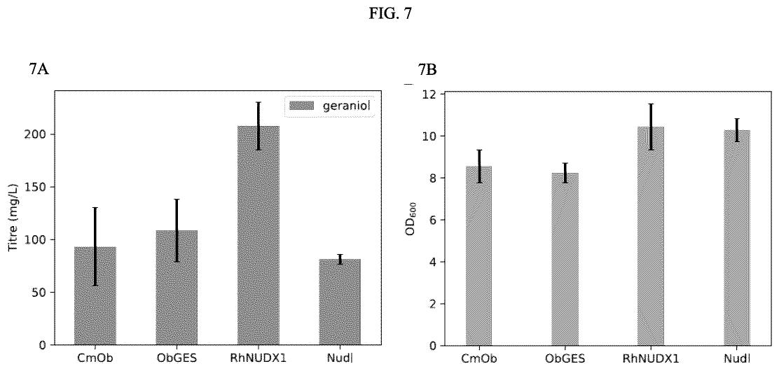

In addition to RhNUDX1 enzyme, the other types of enzymes to produce geraniol were also compared. As the GES from sweet basil (ObGES) has relatively lower Kₘ value than other GESs from Cinnamomum tenuipile and Camptotheca acuminata (Happy tree), ObGES was chosen. Also, NudI from Escherichia coli, which is more over GPP than many other microbial Nudix hydrolases reported, was selected. However, RhNUDX1 were found to outperform ObGES and NudI for geraniol biosynthesis (FIGS. 7A and B). The yield of geraniol using RhNUDX1 almost doubled than the other two enzymes. We also tested N-terminal fused ObGES (Cm-Ob) with the chloramphenicol acetyltransferase leader sequence (CM29*) which was reported to boost the yield of geraniol. However, the increase in geraniol yield was very obvious in our study (FIGS. 7A and B).

Next, the combination of various enzymes with additional GPPS or co-utilization of RhNUDX1 and ObGES was explored. Though the GPPS was optimized in FIG. 3, the GPP supply is speculated to limit the production of geraniol. If so, the expression of additional GPPS could boost the geraniol production. However, in all the designs (RhNUDX1-ispA, ObGES-ispA, or Cb-Ob), additional AgGPPS reduced the geraniol yield by 7-47% (FIGS. 7A and C). The combination of RhNUDX1-ObGES-ispA-AgGPPS resulted in the lowest production of geraniol (32.4 mg/L). The data indicated: 1) oversupply of GPPS was detrimental, instead of beneficial, for geraniol production; 2) the two enzymes (ObGES and RhNUDX1) seemed to compete with each rather than working synergistically. The new combinations led to perturbation of the overall pathway and reduced geraniol production. Thus, the mevalonate pathway was re-optimized, and obtained a new strain (RhNUDX1-AgGPPS*) whose yield (254 mg/L) was higher the strain 32 (or NUDX1-ispA, FIGS. 7C and D).

### Biosynthesis of Geranyl Acetate

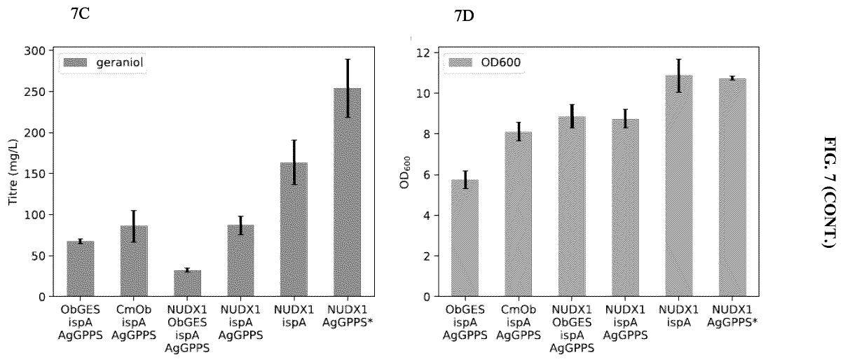

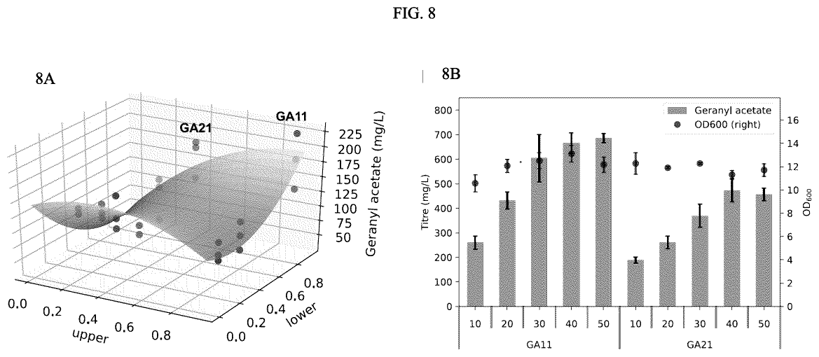

On top of the knowledge learnt from geraniol, the production of geranyl acetate using RhNUDX1 and RhAAT1, the rose alcohol acyltransferase (FIG. 1) was explored. Using the same strategy of pathway balancing, two strains GA11 and GA21 had the highest yield of geranyl acetate (FIG. 8A). Further tuning of the lactose concentration resulted in highest yield of geranyl acetate in strain GA11 (686 mg/L, FIG. 8B), about 1.7 fold higher than the yield of geraniol (254 mg/L). Similar boosting effect was also reported previously that the esterification of geraniol can increase the product yield.

### Batch and Fed-Batch Fermentation of Geraniol and Geranyl Acetate

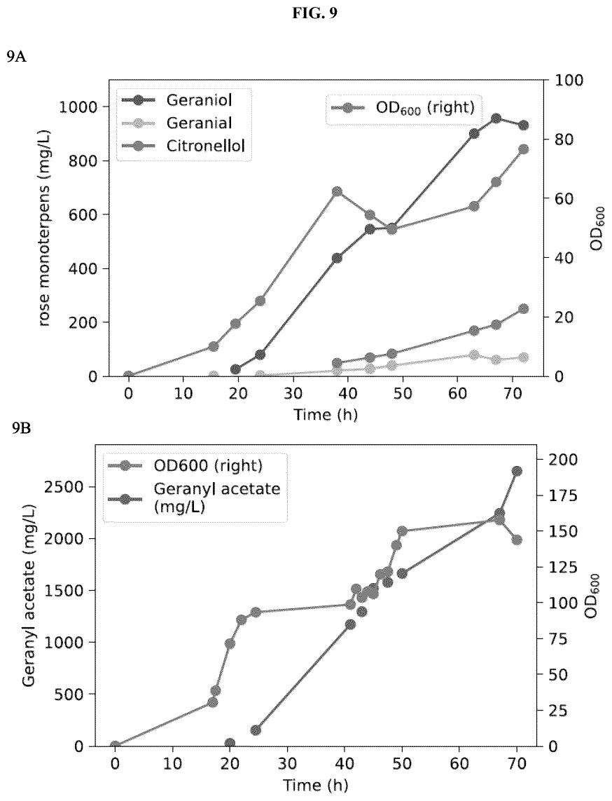

As the auto-induction media led to highest yield of geraniol by minimizing its degradation into other products (geranial and citronellol), a new bioprocess was developed based on the concept of auto-induction media. High-cell-density batch fermentation was explored by increasing the key nutrient supply (carbon, nitrogen and MgSO₄). The medium including 10 g/L glucose, 50 g/L of glycerol, 20 g/L (58 mM) lactose, 6 g/L (NH₄)₂SO₄ and 1.5 g/L MgSO₄ was first tested. Other nutrients were maintained the same as our previously used defined medium (see Material and Methods). In such as batch fermentation, up to an OD₆₀₀ of 77 was achieved, and the geraniol titre reached 957 mg/L in 67 h (FIG. 9A). In addition, the production of citronellol was observed to increase rapidly in the late stage of fermentation and reached 250 mg/L at 72 h. Concurrently, geraniol and geranial dropped slightly from 67 to 72 h. Citronellol was reduced from geraniol/geranial in E. coli, however, the enzyme was still unknown. In the future, identification of the enzyme and deletion of the enzyme from the genome to minimize the formation of citronellol will be explored.

Next, the production of geranyl acetate in a fed-batch fermentation was tested as previously used in the production of viridiflorol and amorphadiene. Within 70 h, the strain GA11 produced 2.65 g/L of geranyl acetate, with an OD₆₀₀ of 144.

Deletion of ackA and Pta Genes

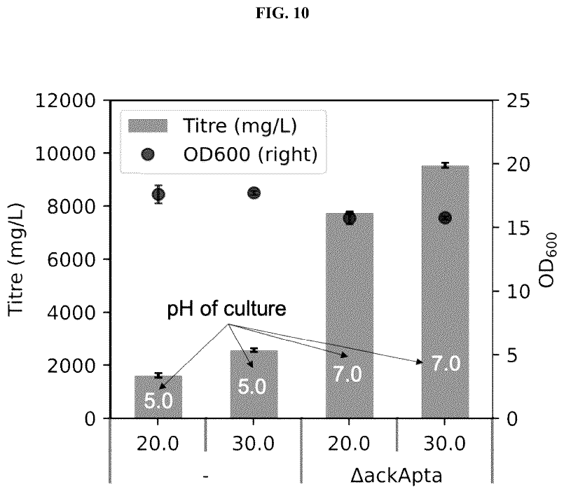

While developing the bioprocesses, lots of acetic acid (up to 10 g/L) was produced along with the geranyl acetate. In E. coli, the main route to produce acetate is through acetate kinase (ackA) and phosphate acetyltransferase (pta). Together, the two enzymes convert 1 acetyl-CoA to 1 acetate and 1 ATP. As the mevalonate and geraniol pathway also started from acetyl-CoA, the acetic acid production competed directly with geraniol biosynthesis. Hence, the deletion of ackA and pta on geranyl acetate bioproduction was evaluated. The deletion of ackA and pta markedly improved the titre of geranyl acetate by 3.7-4.8-fold from 1.6 to 7.8 g/L in the media with 20 g/L glycerol or from 2.6 to 9.5 g/L in the media with 30 g/L glycerol (FIG. 10). In addition, the acetic acid production was greatly reduced as witnessed by pH difference. After 3-day cultivation, the pH values of the wildtype (“−”) dropped from 7.2 to about 5.0, indicating the acetic acid production was pretty high. In constant, the mutant strain (“ΔackApta”) pH remained about 7.0. The carbon yield reached about >24% which is about 2-3 times higher than the best achievement in the literature.

In addition to geranyl acetate, the deletion of ackA and pta also worked for the biosynthesis of geraniol.

### Tryptone Supplementation

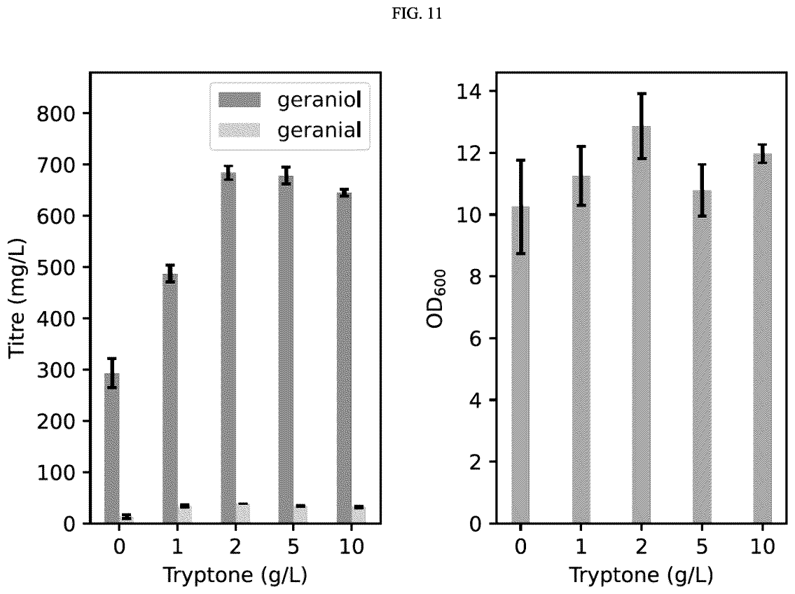

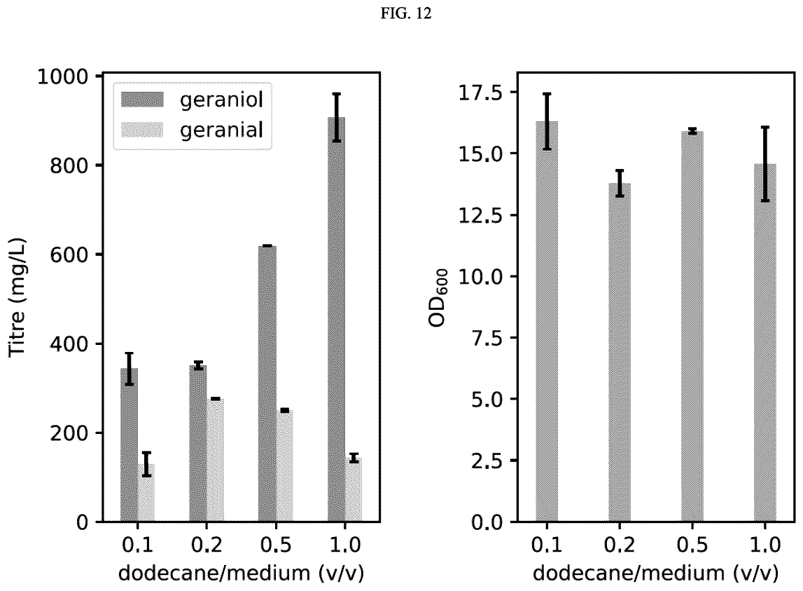

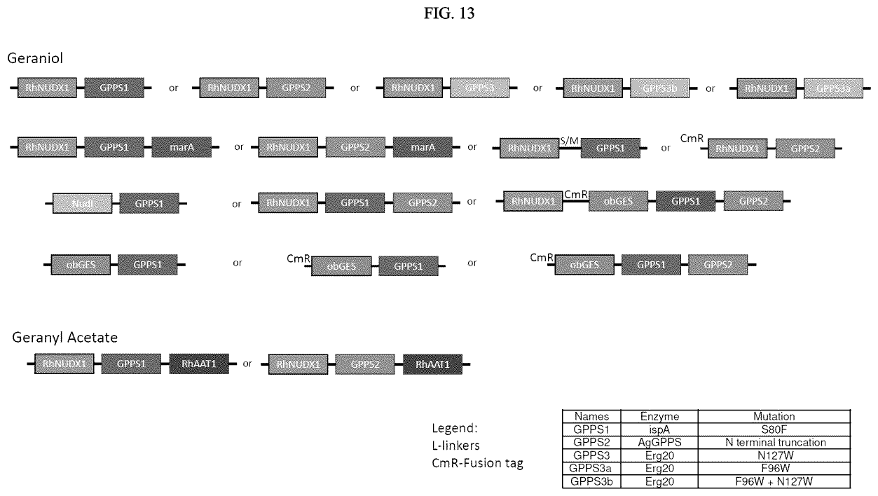

Next, the effect of tryptone supplementation on geraniol production was evaluated. A very positive impact of tryptone supplementation on geraniol production was observed. With supplementation of only 2 g/L of tryptone, the geraniol production increased by 130% from 292 to 684 mg/L (FIG. 11). Further increase of tryptone could not increase the geraniol yield anymore. The change of OD₆₀₀ values was not very significant.

### Organic Effects

While testing the strains in flasks, geraniol production was consistently lower than that in tubes while geranial production was higher than in tubes. The hypothesis was that the extracellular geraniol was not stable and might be oxidized by oxygen to geranial. To test this hypothesis, more organic (dodecane was used here) was used in flasks that might protect geraniol from further oxidation. Therefore, different volumetric ratio of dodecane to media from 0.1 to 1 were evaluated. At the ratio of 0.1-0.2, the effect was not obvious on geraniol, although geranial production was increased from 129 to 276 mg/L. However, as the ratio further increased to 0.5 and 1.0, the production of geraniol was significantly boosted and the formation of geranial was also reduced. The geraniol titre increased to 907 mg/L in flasks with the ratio of 1.0, about 2.6-fold higher than that with the ratio of 0.1-0.2.

TABLE 6
Summary of sequence listing.

| Description | DNA/Protein sequence | SEQ ID NO |
|---|---|---|
| RBS-1 | gagatataataacgagataaggaaaagacaaa | SEQ ID NO: 1 |
| RBS-2 | gccgagatataataacgagataaggaaaagacaaa | SEQ ID NO: 2 |
| RBS-3 | tgccaactttaaaagaggcctaaa | SEQ ID NO: 3 |
| RBS-4 | tttgtttaactttaagaaggagatatacat | SEQ ID NO: 4 |
| RBS-5 | tttgtttaactttaacaaggagggatacat | SEQ ID NO: 5 |
| Polypeptide | MAYSAMATMGYNGMAASCHTLHPTSPLKPF | SEQ ID NO: 6 |
| sequence of | HGASTSLEAFNGEHMGLLRGYSKRKLSSYK | |
| AgGPPS | NPASRSSNATVAQLLNPPQKGKKAVEFDFN | |
| | KYMDSKAMTVNEALNKAIPLRYPQKIYESMR | |
| | YSLLAGGKRVRPVLCIAACELVGGTEELAIPT | |
| | ACAIEMIHTMSLMHDDLPCIDNDDLRRGKPT | |
| | NHKIFGEDTAVTAGNALHSYAFEHIAVSTSKT | |
| | VGADRILRMVSELGRATGSEGVMGGQMVDI | |
| | ASEGDPSIDLQTLEWIHIHKTAMLLECSVVCG | |
| | AIIGGASEIVIERARRYARCVGLLFQVVDDILD | |
| | VTKSSDELGKTAGKDLISDKATYPKLMGLEK | |
| | AKEFSDELLNRAKGELSCFDPVKAAPLLGLA | |
| | DYVAFRQN | |
| Polypeptide | MFDFNKYMDSKAMTVNEALNKAIPLRYPQKI | SEQ ID NO: 7 |
| sequence of | YESMRYSLLAGGKRVRPVLCIAACELVGGTE | |
| AgGPPS truncated | ELAIPTACAIEMIHTMSLMHDDLPCIDNDDLR | |
| from the amino acid | RGKPTNHKIFGEDTAVTAGNALHSYAFEHIAV | |
| positions 2 to 85 | STSKTVGADRILRMVSELGRATGSEGVMGG | |
| | QMVDIASEGDPSIDLQTLEWIHIHKTAMLLEC | |
| | SVVCGAIIGGASEIVIERARRYARCVGLLFQV | |
| | VDDILDVTKSSDELGKTAGKDLISDKATYPKL | |
| | MGLEKAKEFSDELLNRAKGELSCFDPVKAAP | |
| | LLGLADYVAFRQN | |
| Polypeptide | MVLTNKTVISGSKVKSLSSAQSSSSGPSSSS | SEQ ID NO: 8 |
| sequence of FPPS | EEDDSRDIESLDKKIRPLEELEALLSSGNTKQ | |
| isolated from | LKNKEVAALVIHGKLPLYALEKKLGDTTRAVA | |
| Escherichia coli | VRRKALSILAEAPVLASDRLPYKNYDYDRVF | |
| | GACCENVIGYMPLPVGVIGPLVIDGTSYHIPM | |
| | ATTEGCLVASAMRGCKAINAGGGATTVLTKD | |
| | GMTRGPVVRFPTLKRSGACKIWLDSEEGQN | |
| | AIKKAFNSTSRFARLQHIQTCLAGDLLFMRFR | |
| | TTTGDAMGMNMISKGVEYSLKQMVEEYGW | |
| | EDMEVVSVSGNYCTDKKPAAINWIEGRGKS | |
| | VVAEATIPGDVVRKVLKSDVSALVELNIAKNL | |
| | VGSAMAGSVGGFNAHAANLVTAVFLALGQD | |
| | PAQNVESSNCITLMKEVDGDLRISVSMPSIEV | |
| | GTIGGGTVLEPQGAMLDLLGVRGPHATAPG | |
| | TNARQLARIVACAVLAGELSLCAALAAGHLV | |
| | QSHMTHNRKPAEPTKPNNLDATDINRLKDGS | |
| | VTCIKS | |
| Polypeptide | MDFPQQLEACVKQANQALSRFIAPLPFQNTP | SEQ ID NO: 9 |
| sequence of FPPS | VVETMQYGALLGGKRLRPFLVYATGHM | |
| isolated from | FGVSTNTLDAPAAAVECIHAYFLIHDDLPAMD | |
| Escherichia coli | DDDLRRGLPTCHVKFGEANAILAGDALQTLA | |
| comprising a | FSILSDADMPEVSDRDRISMISELASASGIAG | |
| mutation of serine to | MCGGQALDLDAEGKHVPLDALERIHRHKTG | |
| phenylalanine | ALIRAAVRLGALSAGDKGRRALPVLDKYAESI | |
| mutation at amino | GLAFQVQDDILDVVGDTATLGKRQGADQQL | |
| acid position 80 | GKSTYPALLGLEQARKKARDLIDDARQSLKQ | |
| | LAEQSLDTSALEALADYIIQRNK | |
| Polypeptide | MASEKEIRRERFLNVFPKLVEELNASLLAYG | SEQ ID NO: 10 |
| sequence of FPPS | MPKEÅCDWYAHSLNYNTPGGKLNRGLSVVD | |
| isolated from | TYAILSNKTVEQLGQEEYEKVAILGWCIELLQ | |
| Saccharomyces | AYFLVADDMMDKSITRRGQPCWYKVPEVGE | |
| cerevisiae | IAINDAFMLEAAIYKLLKSHFRNEKYYIDITELF | |
| | HEVTFQTELGQLMDLITAPEDKVDLSKFSLKK | |
| | HSFIVTFKTAYYSFYLPVALAMYVAGITDEKD | |
| | LKQARDVLIPLGEYFQIQDDYLDCFGTPEQIG | |
| | KIGTDIQDNKCSWVINKALELASAEQRKTLDE | |
| | NYGKKDSVAEAKCKKIFNDLKIEQLYHEYEES | |
| | IAKDLKAKISQVDESRGFKADVLTAFLNKVYK | |
| | RSK | |
| FPPS isolated from | MASEKEIRRERFLNVFPKLVEELNASLLAYG | SEQ ID NO: 11 |
| Saccharomyces | MPKEACDWYAHSLNYNTPGGKLNRGLSVVD | |
| cerevisiae | TYAILSNKTVEQLGQEEYEKVAILGWCIELLQ | |
| comprising a | AYFLVADDMMDKSITRRGQPCWYKVPEVGE | |
| mutation of | IAIWDAFMLEAAIYKLLKSHFRNEKYYIDITELF | |
| asparagine to | HEVTFQTELGQLMDLITAPEDKVDLSKFSLKK | |
| tryptophan mutation | HSFIVTFKTAYYSFYLPVALAMYVAGITDEKD | |
| at amino acid | LKQARDVLIPLGEYFQIQDDYLDCFGTPEQIG | |
| position 127 | KIGTDIQDNKCSWVINKALELASAEQRKTLDE | |
| | NYGKKDSVAEAKCKKIFNDLKIEQLYHEYEES | |
| | IAKDLKAKISQVDESRGFKADVLTAFLNKVYK | |
| | RSK | |
| FPPS isolated from | MASEKEIRRERFLNVFPKLVEELNASLLAYG | SEQ ID NO: 12 |
| Saccharomyces | MPKEÅCDWYAHSLNYNTPGGKLNRGLSVVD | |
| cerevisiae | TYAILSNKTVEQLGQEEYEKVAILGWCIELLQ | |
| comprising a | AYWLVADDMMDKSITRRGQPCWYKVPEVG | |
| mutation of | EIAINDAFMLEAAIYKLLKSHFRNEKYYIDITEL | |
| phenylalanine to | FHEVTFQTELGQLMDLITAPEDKVDLSKFSLK | |
| tryptophan mutation | KHSFIVTFKTAYYSFYLPVALAMYVAGITDEK | |
| at amino acid | DLKQARDVLIPLGEYFQIQDDYLDCFGTPEQI | |
| position 96 | GKIGTDIQDNKCSWVINKALELASAEQRKTLD | |
| | ENYGKKDSVAEAKCKKIFNDLKIEQLYHEYEE | |
| | SIAKDLKAKISQVDESRGFKADVLTAFLNKVY | |
| | KRSK | |
| FPPS isolated from | MASEKEIRRERFLNVFPKLVEELNASLLAYG | SEQ ID NO: 13 |
| Saccharomyces | MPKEACDWYAHSLNYNTPGGKLNRGLSVVD | |
| cerevisiae | TYAILSNKTVEQLGQEEYEKVAILGWCIELLQ | |
| comprising | AYWLVADDMMDKSITRRGQPCWYKVPEVG | |
| mutations of | EIAIWDAFMLEAAIYKLLKSHFRNEKYYIDITEL | |
| asparagine to | FHEVTFQTELGQLMDLITAPEDKVDLSKFSLK | |
| tryptophan mutation | KHSFIVTFKTAYYSFYLPVALAMYVAGITDEK | |
| at amino acid | DLKQARDVLIPLGEYFQIQDDYLDCFGTPEQI | |
| position 127, and | GKIGTDIQDNKCSWVINKALELASAEQRKTLD | |
| phenylalanine to | ENYGKKDSVAEAKCKKIFNDLKIEQLYHEYEE | |
| tryptophan mutation | SIAKDLKAKISQVDESRGFKADVLTAFLNKVY | |
| at amino acid | KRSK | |
| position 96 | | |
| Polypeptide | MGNETVVVAETAGSIKVAVVVCLLRGQNVLL | SEQ ID NO: 14 |
| sequence of NUDX1 | GRRRSSLGDSTFSLPSGHLEFGESFE | |
| isolated from Rosa | ECAARELKEETDLDIGKIELLTVTNNLFLDEAK | |
| hybrida | PSQYVAVFMRAVLADPRQEPQNIEPEFCDG | |
| | WGWYEWDNLPKPLFWPLDNVVQDGFNPFP | |
| | T | |
| Polypeptide | MSTGEAIPRVAVVVFILNGNSILLGRRRSSIG | SEQ ID NO: 15 |
| sequence of NUDX1 | NST | |
| isolated from | FALPGGHLEFGESFEECAAREVMEETGLKIE | |
| Arabidopsis thaliana | KMKLLTVTNNVFKEAPTPSHYVSVSIRAVLVD | |
| | PSQEPKNMEPEKCEGWDWYDWENLPKPLF | |
| | WPLEKLFGSGFNPFTHGGGD | |
| Nucleic acid | atgCAGCACATGGAGGAGAGCTCTTCCAAG | SEQ ID NO: 16 |
| sequence of GES | CGCCGTGAATACTTGCTGGAGGAAACGAC | |
| isolated from | TCGCAAACTCCAGCGCAACGATACCGAAT | |
| Ocimum basilicum | CCGTGGAAAAACTGAAACTCATTGATAACA | |
| (ObGES) | TCCAGCAACTGGGCATCGGTTATTACTTCG | |
| | AGGACGCAATTAACGCTGTGCTGCGTTCTC | |
| | CGTTCTCTACAGGTGAAGAAGATCTGTTCA | |
| | CCGCCGCTCTGCGTTTCCGTCTGCTGCGT | |
| | CACAACGGCATTGAGATCTCCCCGGAAAT | |
| | CTTCCTGAAGTTTAAAGACGAACGTGGTAA | |
| | ATTCGACGAGTCCGATACCCTCGGCCTGC | |
| | TGAGCCTGTACGAAGCTTCCAACCTGGGT | |
| | GTTGCAGGTGAAGAAATTCTGGAAGAAGC | |
| | GATGGAATTTGCCGAGGCACGCCTGCGCC | |
| | GCTCGCTGTCCGAACCGGCCGCGCCGCT | |
| | GCATGGCGAAGTGGCGCAGGCTCTTGACG | |
| | TCCCGCGCCATCTGCGTATGGCGCGCCTG | |
| | GAAGCACGTCGCTTTATCGAACAGTACGG | |
| | CAAACAGTCTGATCATGACGGTGACCTCCT | |
| | GGAACTGGCTATTCTGGATTATAACCAGGT | |
| | GCAGGCGCAGCACCAGTCCGAACTGACTG | |
| | AAATCATCCGTTGGTGGAAAGAGTTGGGA | |
| | CTTGTCGATAAACTGTCCTTCGGCCGCGAC | |
| | CGTCCGCTGGAATGTTTCCTGTGGACTGTA | |
| | GGCCTGCTGCCAGAACCGAAGTACTCCTC | |
| | TGTACGTATCGAGCTCGCTAAAGCGATCTC | |
| | CATCTTATTGGTCATTGATGATATTTTCGAT | |
| | ACCTACGGTGAAATGGATGATCTGATCCTG | |
| | TTCACCGATGCTATCCGTCGTTGGGATCTG | |
| | GAAGCGATGGAAGGTCTGCCGGAATATAT | |
| | GAAAATCTGCTACATGGCCCTGTATAACAC | |
| | CACGAACGAAGTTTGCTACAAAGTACTGCG | |
| | TGATACCGGTCGTATCGTACTTCTGAACCT | |
| | CAAATCCACATGGATTGACATGATTGAGGG | |
| | TTTCATGGAAGAAGCGAAATGGTTCAACGG | |
| | CGGTTCGGCACCGAAACTGGAGGAGTACA | |
| | TCGAAAATGGTGTGTCTACCGCAGGCGCA | |
| | TACATGGCCTTTGCCCACATTTTCTTCCTG | |
| | ATTGGCGAAGGCGTTACGCACCAGAACTC | |
| | CCAGCTGTTCACCCAAAAACCGTACCCGAA | |
| | AGTGTTTAGCGCCGCCGGTCGCATCCTGC | |
| | GTCTGTGGGATGATCTGGGCACCGCAAAA | |
| | GAAGAGCAGGAACGTGGTGATCTGGCGTC | |
| | CTGCGTTCAGCTGTTTATGAAGGAAAAATC | |
| | CCTGACTGAAGAAGAAGCGCGTTCTCGTAT | |
| | CCTGGAAGAAATTAAAGGTCTGTGGCGCG | |
| | ATTTAAACGGCGAACTGGTGTACAACAAAA | |
| | ACCTGCCGCTTTCCATCATCAAAGTTGCGC | |
| | TGAATATGGCGCGCGCGTCTCAAGTTGTAT | |
| | ACAAACACGATCAGGATACGTATTTCAGCT | |
| | CCGTAGATAACTACGTAGACGCTCTGTTTT | |
| | TTACTCAGTGA | |
| Linker sequence of | GGGGSGGPGS | SEQ ID NO: 17 |
| the engineered | | |
| fusion protein | | |
| Linker sequence of | GGGGSGGGGSGGGGSGGPGS | SEQ ID NO: 18 |
| the engineered | | |
| fusion protein | | |
| Nucleic acid | atgggtaacgtgttacaagccgggctggggcaaaatccggcg | SEQ ID NO: 19 |
| sequence of atoB | cgtcaggcactgttaaaaagcgggctggcagaaacggtgtgc | |
| gene | ggattcacggtcaataaagtatgtggttcgggtcttaaaagtgtg | |
| | gcgcttgccgcccaggccattcaggcaggtcaggcgcagag | |
| | cattgtggcggggggtatggaaaatatgagtttagccccctact | |
| | tactcgatgcaaaagcacgctctggttatcgtcttggagacgga | |
| | caggtttatgacgtaatcctgcgcgatggcctgatgtgcgccac | |
| | ccatggttatcatatggggattaccgccgaaaacgtggctaaa | |
| | gagtacggaattacccgtgaaatgcaggatgaactggcgcta | |
| | cattcacagcgtaaagcggcagccgcaattgagtccggtgctt | |
| | ttacagccgaaatcgtcccggtaaatgttgtcactcgaaagaa | |
| | aaccttcgtcttcagtcaagacgaattcccgaaagcgaattca | |
| | acggctgaagcgttaggtgcattgcgcccggccttcgataaag | |
| | caggaacagtcaccgctgggaacgcgtctggtattaacgacg | |
| | gtgctgccgctctggtgattatggaagaatctgcggcgctggca | |
| | gcaggccttacccccctggctcgcattaaaagttatgccagcg | |
| | gtggcgtgccccccgcattgatgggtatggggccagtacctgc | |
| | cacgcaaaaagcgttacaactggcggggctgcaactggcgg | |
| | atattgatctcattgaggctaatgaagcatttgctgcacagttcctt | |
| | gccgttgggaaaaacctgggctttgattctgagaaagtgaatgt | |
| | caacggcggggccatcgcgctcgggcatcctatcggtgccag | |
| | tggtgctcgtattctggtcacactattacatgccatgcaggcacg | |
| | cgataaaacgctggggctggcaacactgtgcattggcggcgg | |
| | tcagggaattgcgatggtgattgaacggttgaattaa | |
| Nucleic acid | atgaaactctcaactaaactttgttggtgtggtattaaaggaaga | SEQ ID NO: 20 |
| sequence of hmgS | cttaggccgcaaaagcaacaacaattacacaatacaaacttg | |
| gene | caaatgactgaactaaaaaaacaaaagaccgctgaacaaa | |
| | aaaccagacctcaaaatgtcggtattaaaggtatccaaatttac | |
| | atcccaactcaatgtgtcaaccaatctgagctagagaaatttga | |
| | tggcgtttctcaaggtaaatacacaattggtctgggccaaacca | |
| | acatgtcttttgtcaatgacagagaagatatctactegatgtccct | |
| | aactgttttgtctaagttgatcaagagttacaacatcgacaccaa | |
| | caaaattggtagattagaagtcggtactgaaactctgattgaca | |
| | agtccaagtctgtcaagtctgtcttgatgcaattgtttggtgaaaa | |
| | cactgacgtcgaaggtattgacacgcttaatgcctgttacggtg | |
| | gtaccaacgcgttgttcaactctttgaactggattgaatctaacg | |
| | catgggatggtagagacgccattgtagtttgcggtgatattgcc | |
| | atctacgataagggtgccgcaagaccaaccggtggtgccggt | |
| | actgttgctatgtggatcggtcctgatgctccaattgtatttgactct | |
| | gtaagagcttcttacatggaacacgcctacgatttttacaagcc | |
| | agatttcaccagcgaatatccttacgtcgatggtcatttttcattaa | |
| | cttgttacgtcaaggctcttgatcaagtttacaagagttattccaa | |
| | gaaggctatttctaaagggttggttagcgatcccgctggttcgg | |
| | atgctttgaacgttttgaaatatttcgactacaacgttttccatgttc | |
| | caacctgtaaattggtcacaaaatcatacggtagattactatat | |
| | aacgatttcagagccaatcctcaattgttcccagaagttgacgc | |
| | cgaattagctactcgcgattatgacgaatctttaaccgataaga | |
| | acattgaaaaaacttttgttaatgttgctaagccattccacaaag | |
| | agagagttgcccaatctttgattgttccaacaaacacaggtaac | |
| | atgtacaccgcatctgtttatgccgcctttgcatctctattaaacta | |
| | tgttggatctgacgacttacaaggcaagcgtgttggtttattttctt | |
| | acggttccggtttagctgcatctctatattcttgcaaaattgttggtg | |
| | acgtccaacatattatcaaggaattagatattactaacaaatta | |
| | gccaagagaatcaccgaaactccaaaggattacgaagctgc | |
| | catcgaattgagagaaaatgcccatttgaagaagaacttcaa | |
| | acctcaaggttccattgagcatttgcaaagtggtgtttactacttg | |
| | accaacatcgatgacaaatttagaagatcttacgatgttaaaa | |
| | aataa | |
| Nucleic acid | atgagcttaccgttcctgacttcggcaccgggcaaagttatcatt | SEQ ID NO: 21 |
| sequence of mevK | ttcggcgagcactctgctgtttacaacaaaccggcagttgcgg | |
| gene | cctccgtatctgcactgcgcacttatctgctgatctctgaaagctc | |
| | cgccccggatactattgaactggactttccggacatttcctttaac | |
| | cacaaatggagcattaacgactttaacgcgatcactgaagatc | |
| | aggtaaactcccagaaactggcaaaagcacagcaggctac | |
| | cgatggtctgagccaggaactggtgtccctcctcgatcctttgct | |
| | ggctcaactctcggaatcgttccattaccatgctgctttctgttttct | |
| | gtatatgtttgtttgcctctgcccgcacgcgaaaaacatcaaatt | |
| | ctctctgaaatcgactctgccgattggtgccggcctgggttcgtc | |
| | cgcatctatttccgtttccctggcgctggccatggcctatctgggc | |
| | ggtctgatcggttccaacgacctggaaaaactctoggaaaac | |
| | gataagcacatcgttaaccagtgggcgttcatcggtgaaaaat | |
| | gtatccacggtaccccatccggtatcgataatgcggttgctacct | |
| | acggtaacgcgttactgttcgaaaaagattctcataacggtact | |
| | atcaacactaacaacttcaaatttttggacgattttccggcgattc | |
| | cgatgatcctgacttacacccgcatcccgcgtagcaccaagg | |
| | atctggttgcacgcgttcgtgtactcgtgaccgaaaaattcccg | |
| | gaggttatgaaaccgatcctggatgcaatgggtgagtgcgcg | |
| | ctgcagggattagaaatcatgaccaaactgtcgaagtgtaaa | |
| | ggtacggacgacgaagctgttgaaacaaacaacgaactgta | |
| | tgaacagctgctggaactgatccgtatcaaccacggcctgctg | |
| | gtcagcattggtgtgagccacccgggcctggaactgattaaaa | |
| | atctttcggatgacctgcgcattggttctaccaagctgactggtg | |
| | ctggcggcggtggctgttctctgaccctcctgcgtcgcgatatta | |
| | cccaggaacaaatcgactcgttcaaaaaaaaactgcaggat | |
| | gacttttcttatgaaaccttcgaaaccgacctgggcggtaccgg | |
| | ctgttgtctcctgtccgccaaaaacttgaacaaagatctgaaaa | |
| | tcaaatctctcgtotttcagctgtttgaaaacaaaactaccacca | |
| | aacaacaaattgatgacctgctgctcccgggcaacaccaactt | |
| | accgtggacttcctaa | |
| Nucleic acid | atgtctgagcttcgcgctttctccgctccgggcaaggccctgctg | SEQ ID NO: 22 |
| sequence of pmk | gccggaggctatctggtgctggacaccaaatatgaagcgtttgt | |
| gene | agttggtctgtctgcccgtatgcatgcggtcgcgcacccgtatg | |
| | ggtcgctgcagggttcagataaattcgaggtgcgagtgaaaa | |
| | gcaaacagtttaaagacggtgagtggctgtatcacattagccc | |
| | gaaatctggttttatcccggtatccatcggcggttccaaaaacc | |
| | cgttcattgaaaaagttattgccaacgttttctcctattttaaaccta | |
| | acatggatgactactgtaaccgtaacctgttcgtgattgatattttc | |
| | tctgacgatgcttaccattcccaggaagacagcgttacggaac | |
| | accgtggcaaccgtcgtctgtcgtttcattcccaccgtatcgaag | |
| | aagttccgaagactggcctgggtagctctgcaggcctggttac | |
| | cgtcctgactactgctctggcctctttttttgtgtcggatctggaaa | |
| | acaacgttgacaaatatcgtgaggtaattcataacctggctcag | |
| | gtcgcacactgccaggcgcagggcaaaatcggctccggtttc | |
| | gatgttgctgcggcagcttatggctccattegttaccgtcgcttcc | |
| | cgcctgctctgatctcaaacctgccggatattggtagogcaacc | |
| | tacggatcgaagctggctcacctggtggatgaggaagattgg | |
| | aatatcaccattaaatctaaccacctgccgtctggcctgaccct | |
| | gtggatgggtgatatcaaaaacggctctgaaaccgtcaaact | |
| | ggtacagaaagttaagaattggtatgattctcacatgccggaat | |
| | ccctgaaaatctacaccgagctggatcacgcgaactcacgttt | |
| | catggacggtctgtccaaactggaccgtctgcacgaaaccca | |
| | cgatgattacagcgaccagatcttcgaatctctggaacgtaac | |
| | gactgcacctgtcaaaaatacccggaaatcaccgaagttcgt | |
| | gacgcggtagcgaccatccgccgctctttccgtaaaatcacta | |
| | aggaaagcggcgctgacatcgaaccgccggttcagacctcc | |
| | ctgctggacgattgccagactctgaaaggggtgctgacctgtct | |
| | gattccgggtgcgggggttatgacgctatcgcagtgatcacga | |
| | aacaggatgtagacctgcgtgcgcagactgcaaacgacaaa | |
| | cgttttagcaaagtacaatggctggatgttacccaggcggattg | |
| | gggtgttcgtaaagaaaaggaccctgaaacctacctggataa | |
| | ataa | |
| Nucleic acid | atgaccgtttacacagcatccgttaccgcacccgtcaacatcg | SEQ ID NO: 23 |
| sequence of pmd | caacccttaagtattgggggaaaagggacacgaagttgaatc | |
| gene | tgcccaccaattcgtccatatcagtgactttatcgcaagatgacc | |
| | tcagaacgttgacctctgcggctactgcacctgagtttgaacgc | |
| | gacactttgtggttaaatggagaaccacacagcatcgacaatg | |
| | aaagaactcaaaattgtctgcgcgacctacgccaattaagaa | |
| | aggaaatggaatcgaaggacgcctcattgcccacattatctca | |
| | atggaaactccacattgtctccgaaaataactttcctacagcag | |
| | ctggtttagcttcctccgctgctggctttgctgcattggtctctgcaa | |
| | ttgctaagttataccaattaccacagtcaacttcagaaatatccc | |
| | gtatagcaagaaaggggtctggttcagcttgtagatcgttgtttg | |
| | gcggatacgtggcctgggaaatgggaaaagctgaagatggt | |
| | catgattccatggcagtacaaatcgcagacagctctgactggc | |
| | ctcagatgaaagcttgtgtcttagtcgtcagcgatattaaaaag | |
| | gatgtgagttccactcagggtatgcaattgaccgtggcaacctc | |
| | cgaactatttaaagaaagaattgaacatgtcgtaccaaagag | |
| | atttgaagtcatgcgtaaagccattgttgaaaaagatttcgcca | |
| | cctttgcaaaggaaacaatgatggattccaactctttccatgcc | |
| | acatgtttggactctttccctccaatattctacatgaatgacacttc | |
| | caagcgtatcatcagttggtgccacaccattaatcagttttacgg | |
| | agaaacaatcgttgcatacacgtttgatgcaggtccaaatgctg | |
| | tgttgtactacttagctgaaaatgagtcgaaactctttgcatttatc | |
| | tataaattgtttggctctgttcctggatgggacaagaaatttacta | |
| | ctgagcagcttgaggctttcaaccatcaatttgaatcatctaactt | |
| | tactgcacgtgaattggatcttgagttgcaaaaggatgttgcca | |
| | gagtgattttaactcaagtcggttcaggcccacaagaaacaaa | |
| | cgaatctttgattgacgcaaagactggtctaccaaaggaataa | |
| Nucleic acid | atgcatcatcatcaccatcacgagctccaaacggaacacgtc | SEQ ID NO: 24 |
| sequence of idi | attttattgaatgcacagggagttcccacgggtacgctggaaa | |
| gene | agtatgccgcacacacggcagacacccgcttacatctcgcgtt | |
| | ctccagttggctgtttaatgccaaaggacaattattagttacccg | |
| | ccgcgcactgagcaaaaaagcatggcctggcgtgtggacta | |
| | actcggtttgtgggcacccacaactgggagaaagcaacgaa | |
| | gacgcagtgatccgccgttgccgttatgagcttggcgtggaaat | |
| | tacgcctcctgaatctatctatcctgactttcgctaccgcgccacc | |
| | gatccgagtggcattgtggaaaatgaagtgtgtccggtatttgc | |
| | cgcacgcaccactagtgcgttacagatcaatgatgatgaagtg | |
| | atggattatcaatggtgtgatttagcagatgtattacacggtattg | |
| | atgccacgccgtgggcgttcagtccgtggatggtgatgcaggc | |
| | gacaaatcgcgaagccagaaaacgattatctgcatttaccca | |
| | gcttaaataa | |
| Nucleic acid | ATGcatcaccatcaccatcacGGTAACGAAACTGT | SEQ ID NO: 25 |
| sequence of | GGTAGTCGCAGAAACCGCGGGTAGCATCA | |
| RhNUDX1 gene | AAGTTGCCGTTGTGGTTTGTCTGTTGCGTG | |
| | GTCAAAACGTGCTCCTGGGTCGTCGTCGC | |
| | TCCTCTCTGGGTGACAGCACCTTCTCTCTG | |
| | CCGAGCGGCCACTTAGAATTTGGTGAATCT | |
| | TTCGAAGAATGTGCCGCCCGTGAGCTGAA | |
| | AGAAGAAACGGATCTGGACATCGGTAAAAT | |
| | CGAGCTGCTGACCGTAACCAACAACCTGTT | |
| | CCTGGATGAAGCTAAACCGTCCCAGTATGT | |
| | TGCAGTGTTCATGCGTGCTGTTCTGGCCG | |
| | ATCCGCGTCAGGAGCCGCAGAACATTGAA | |
| | CCCGAATTCTGCGACGGCTGGGGTTGGTA | |
| | CGAATGGGATAACTTACCGAAGCCACTGTT | |
| | CTGGCCGCTCGACAACGTGGTCCAGGATG | |
| | GCTTCAACCCGTTCCCGACTTAA | |
| Nucleic acid | gtgcgacaacggactattgtatgccctttgattcaaaatgatggt | SEQ ID NO: 26 |
| sequence of Nudl | gcttatttgctgtgtaaaatggccgacgatcgcggcgttttcccc | |
| gene | ggtcaatgggcgatttcgggtggcggcgtggagcctggcgaa | |
| | cgaattgaagaggcactacgccgcgaaattcgcgaagaact | |
| | gggagaacagctgcttttgacagaaatcacgccgtggaccttc | |
| | agcgatgatattcgcaccaagacgtatgcagatggtcgcaag | |
| | gaagagatttatatgatttacctgatttttgactgcgtttctgccaa | |
| | ccgagaagtgaaaataaacgaagagtttcaggactacgcgt | |
| | gggtaaaacctgaagatctggtgcattatgatttgaatgtcgcc | |
| | acccgaaaaacgttacgtttgaaaggtcttctgtaa | |
| Polypeptide | MVLTNKTVIS GSKVKSLSSA QSSSSGPSSS | SEQ ID NO: 27 |
| sequence of | SEEDDSRDIE SLDKKIRPLE ELEALLSSGN | |
| truncated hmgR | TKQLKNKEVA ALVIHGKLPL YALEKKLGDT | |
| gene | TRAVAVRRKA LSILAEAPVL ASDRLPYKNY | |
| | DYDRVFGACC ENVIGYMPLP VGVIGPLVID | |
| | GTSYHIPMAT TEGCLVASAM RGCKAINAGG | |
| | GATTVLTKDG MTRGPVVRFP TLKRSGACKI | |
| | WLDSEEGQNA IKKAFNSTSR FARLQHIQTC | |
| | LAGDLLFMRF RTTTGDAMGM | |
| | NMISKGVEYS LKQMVEEYGW | |
| | EDMEVVSVSG NYCTDKKPAA | |
| | INWIEGRGKS VVAEATIPGD VVRKVLKSDV | |
| | SALVELNIAK NLVGSAMAGS VGGFNAHAAN | |
| | LVTAVFLALG QDPAQNVESS NCITLMKEVD | |
| | GDLRISVSMP SIEVGTIGGG TVLEPQGAML | |
| | DLLGVRGPHA TAPGTNARQL ARIVACAVLA | |
| | GELSLCAALA AGHLVQSHMT HNRKPAEPTK | |
| | PNNLDATDIN RLKDGSVTCI KS | |
| Nucleic acid | atggttttaaccaataaaacagtcatttctggatcgaaagtcaa | SEQ ID NO: 28 |
| sequence of | aagtttatcatctgcgcaatcgagctcatcaggaccttcatcatc | |
| truncated hmgR | tagtgaggaagatgattcccgcgatattgaaagcttggataag | |
| gene | aaaatacgtcctttagaagaattagaagcattattaagtagtgg | |
| | aaatacaaaacaattgaagaacaaagaggtcgctgccttggt | |
| | tattcacggtaagttacctttgtacgctttggagaaaaaattaggt | |
| | gatactacgagagcggttgcggtacgtaggaaggctctttcaa | |
| | ttttggcagaagctcctgtattagcatctgatcgtttaccatataaa | |
| | aattatgactacgaccgcgtatttggcgcttgttgtgaaaatgtta | |
| | taggttacatgcctttgcccgttggtgttataggccccttggttatc | |
| | gatggtacatcttatcatataccaatggcaactacagagggttg | |
| | tttggtagcttctgccatgcgtggctgtaaggcaatcaatgctgg | |
| | cggtggtgcaacaactgttttaactaaggatggtatgacaaga | |
| | ggcccagtagtccgtttcccaactttgaaaagatctggtgcctgt | |
| | aagatatggttagactcagaagagggacaaaacgcaattaa | |
| | aaaagcttttaactctacatcaagatttgcacgtctgcaacatatt | |
| | caaacttgtctagcaggagatttactcttcatgagatttagaaca | |
| | actactggtgacgcaatgggtatgaatatgatttctaaaggtgtc | |
| | gaatactcattaaagcaaatggtagaagagtatggctgggaa | |
| | gatatggaggttgtctccgtttctggtaactactgtaccgacaaa | |
| | aaaccagctgccatcaactggatcgaaggtcgtggtaagagt | |
| | gtcgtcgcagaagctactattcctggtgatgttgtcagaaaagt | |
| | gttaaaaagtgatgtttccgcattggttgagttgaacattgctaag | |
| | aatttggttggatctgcaatggctgggtctgttggtggatttaacg | |
| | cacatgcagctaatttagtgacagctgttttcttggcattaggaca | |
| | agatcctgcacaaaatgttgaaagttccaactgtataacattga | |
| | tgaaagaagtggacggtgatttgagaatttccgtatccatgcca | |
| | tccatcgaagtaggtaccatcggtggtggtactgttctagaacc | |
| | acaaggtgccatgttggacttattaggtgtaagaggcccgcat | |
| | gctaccgctcctggtaccaacgcacgtcaattagcaagaata | |
| | gttgcctgtgccgtcttggcaggtgaattatccttatgtgctgccct | |
| | agcagccggccatttggttcaaagtcatatgacccacaacag | |
| | gaaacctgctgaaccaacaaaacctaacaatttggacgcca | |
| | ctgatataaatcgtttgaaagatgggtccgtcacctgcattaaat | |
| | cctaa | |
| Nucleic acid | aaattaatacgactcactataggggaattgtgagcggataaca | SEQ ID NO: 29 |
| sequence of T7 | | |
| promoter | | |
| Nucleic acid | aaattaatacgactcactaatggggaattgtgagcggataaca | SEQ ID NO: 30 |
| sequence of TM1 | | |
| promoter | | |
| Nucleic acid | aaattaatacgactcactcgaggggaattgtgagcggataac | SEQ ID NO: 31 |
| sequence of TM2 | a | |
| promoter | | |
| Nucleic acid | aaattaatacgactcactataaaggaattgtgagcggataaca | SEQ ID NO: 32 |
| sequence of TM3 | | |
| promoter | | |
| Nucleic acid | aaattaatacgactcactaTAGGGgaattgtgagcggataa | SEQ ID NO: 33 |
| sequence of TV1 | ca | |
| promoter | | |
| Nucleic acid | aaattaatacgactcactaCAGACgaattgtgagcggataa | SEQ ID NO: 34 |
| sequence of TV2 | ca | |
| promoter | | |
| Nucleic acid | aaattaatacgactcactaGCGGAgaattgtgagcggata | SEQ ID NO: 35 |
| sequence of TV3 | aca | |
| promoter | | |
| Nucleic acid | aaattaatacgactcactaacaccgaattgtgagcggataaca | SEQ ID NO: 36 |
| sequence of TV4 | | |
| promoter | | |
| Oligo sequence of | GGTAGTGGCGGCGGTGGCAGCGGTGGCC | SEQ ID NO: 37 |
| spk2m1-f | CGGGCAGCatgcatcatcatcaccatcacgag | |
| Oligo sequence of | CACCGCCGCCACTACCACCGCCGCCGCTG | SEQ ID NO: 38 |
| spk2m1-r | CCACCGCCACCAGTCGGGAACGGGTTGAA | |
| | GC | |
| Oligo sequence of | GCGGTGGCCCGGGCAGCatgcatcatcatcaccat | SEQ ID NO: 39 |
| spk2s1-f | cacgag | |
| Oligo sequence of | GCCCGGGCCACCGCTGCCACCGCCACCA | SEQ ID NO: 40 |
| spk2s1-r | GTCGGGAACGGGTTGAAGC | |
| Oligo sequence of | TATGCCGCAAAAGAAGCGGG | SEQ ID NO: 41 |
| yjbB gRNA | | |
| Oligo sequence of | CACGAATGCGGAACGGTTCA | SEQ ID NO: 42 |
| tnaA gRNA | | |
| RBS sequence of | Gagatataataacgagataaggaaaagacaaa | SEQ ID NO: 43 |
| AgGPPS (GPPS2) | | |
| (RBS-1) | | |
| Protein sequence | Atgttcgatttcaacaaatacatggacagcaaagccatga | SEQ ID NO: 44 |
| (N-term 35 NT) of | | |
| AgGPPS (GPPS2) | | |
| (RBS-1) | | |
| RBS sequence of | gccgagatataataacgagataaggaaaagacaaa | SEQ ID NO: 45 |
| AgGPPS (GPPS2) | | |
| (RBS-2) | | |
| Protein sequence | Atgttcgatttcaacaaatacatggacagcaaagccatga | SEQ ID NO: 46 |
| (N-term 35 NT) of | | |
| AgGPPS (GPPS2) | | |
| (RBS-2) | | |
| RBS sequence of | Tgccaactttaaaagaggcctaaa | SEQ ID NO: 47 |
| ERG20_N127W | | |
| (GPPS3) (RBS-3) | | |
| Protein sequence | Atggcttcagaaaaagaaattaggagagagagattcttga | SEQ ID NO: 48 |
| (N-term 35 NT) of | | |
| ERG20_N127W | | |
| (GPPS3) (RBS-3) | | |
| RBS sequence of | gccgagatataataacgagataaggaaaagacaaa | SEQ ID NO: 49 |
| ERG20_N127W | | |
| (GPPS3) (RBS-2) | | |
| Protein sequence | Atggcttcagaaaaagaaattaggagagagagattcttga | SEQ ID NO: 50 |
| (N-term 35 NT) of | | |
| ERG20_N127W | | |
| (GPPS3) (RBS-2) | | |
| RBS sequence of | Tgccaactttaaaagaggcctaaa | SEQ ID NO: 51 |
| ERG20_96_127 | | |
| (GPPS3b) (RBS-3) | | |
| Protein sequence | Atggcttcagaaaaagaaattaggagagagagattcttga | SEQ ID NO: 52 |
| (N-term 35 NT) of | | |
| ERG20_96_127 | | |
| (GPPS3b) (RBS-3) | | |
| RBS sequence of | gccgagatataataacgagataaggaaaagacaaa | SEQ ID NO: 53 |
| ERG20_96_127 | | |
| (GPPS3b) (RBS-2) | | |
| Protein sequence | Atggcttcagaaaaagaaattaggagagagagattcttga | SEQ ID NO: 54 |
| (N-term 35 NT) of | | |
| ERG20_96_127 | | |
| (GPPS3b) (RBS-2) | | |
| RBS sequence of | Tttgtttaactttaagaaggagatatacat | SEQ ID NO: 55 |
| IspA (GPPS1) | | |
| (RBS-4) | | |
| Protein sequence | Atgcatcatcatcaccatcacgagctcgactttccgcago | SEQ ID NO: 56 |
| (N-term 35 NT) of | | |
| IspA (GPPS1) | | |
| (RBS-4) | | |
| RBS sequence of | Tttgtttaactttaacaaggagggatacat | SEQ ID NO: 57 |
| IspA (GPPS1) | | |
| (RBS-5) | | |
| Protein sequence | Atgcatcatcatcaccatcacgagctcgactttccgcagc | SEQ ID NO: 58 |
| (N-term 35 NT) of | | |
| IspA (GPPS1) | | |
| (RBS-5) | | |
| RBS sequence of | tttgtttaactttaagaaggagatatacat | SEQ ID NO: 59 |
| RBS-SAR (hmgs (S)) | | |
| RBS sequence of | atcgattcacaacacgttgatgaagtgatt | SEQ ID NO: 60 |
| RBS-SAR (atoB (A)) | | |
| RBS sequence of | ctcgagaataaggaggtaagtc | SEQ ID NO: 61 |
| RBS-SAR (thmgR (R)) | | |
| RBS sequence of | aacacaaacagaggggaaaaaa | SEQ ID NO: 62 |
| RBS-MPPI (MK (M)) | | |
| RBS sequence of | tcactagacacatoggataaggaggtacct | SEQ ID NO: 63 |
| RBS-MPPI (PMK (P)) | | |
| RBS sequence of | tccttatcattccatoccataagagaggcact | SEQ ID NO: 64 |
| RBS-MPPI (MVD | | |
| (P)) | | |
| RBS sequence of | ccccactcaggaaaataataagagaggacct | SEQ ID NO: 65 |
| RBS-MPPI (idi (I)) | | |
| Polypeptide | MPPLFKGLKQMAKPIAYVSRFSAKRPIHIILFS | SEQ ID NO: 66 |
| sequence of hmgR | LIISAFAYLSVIQYYFNGWQLDSNSVFETAPN | |
| | KDSNTLFQECSHYYRDSSLDGWVSITAHEAS | |
| | ELPAPHHYYLLNLNFNSPNETDSIPELANTVF | |
| | EKDNTKYILQEDLSVSKEISSTDGTKWRLRS | |
| | DRKSLFDVKTLAYSLYDVFSENVTQADPFDV | |
| | LIMVTAYLMMFYTIFGLFNDMRKTGSNFWLS | |
| | ASTVVNSASSLFLALYVTQCILGKEVSALTLF | |
| | EGLPFIVVVVGFKHKIKIAQYALEKFERVGLSK | |
| | RITTDEIVFESVSEEGGRLIQDHLLCIFAFIGC | |
| | SMYAHQLKTLTNFCILSAFILIFELILTPTFYSAI | |
| | LALRLEMNVIHRSTIIKQTLEEDGVVPSTARIIS | |
| | KAEKKSVSSFLNLSVVVIIMKLSVILLFVFINFY | |
| | NFGANWVNDAFNSLYFDKERVSLPDFITSNA | |
| | SENFKEQAIVSVTPLLYYKPIKSYQRIEDMVLL | |
| | LLRNVSVAIRDRFVSKLVLSALVCSAVINVYLL | |
| | NAARIHTSYTADQLVKTEVTKKSFTAPVQKA | |
| | STPVLTNKTVISGSKVKSLSSAQSSSSGPSS | |
| | SSEEDDSRDIESLDKKIRPLEELEALLSSGNT | |
| | KQLKNKEVAALVIHGKLPLYALEKKLGDTTRA | |
| | VAVRRKALSILAEAPVLASDRLPYKNYDYDR | |
| | VFGACCENVIGYMPLPVGVIGPLVIDGTSYHI | |
| | PMATTEGCLVASAMRGCKAINAGGGATTVLT | |
| | KDGMTRGPVVRFPTLKRSGACKIWLDSEEG | |
| | QNAIKKAFNSTSRFARLQHIQTCLAGDLLFMR | |
| | FRTTTGDAMGMNMISKGVEYSLKQMVEEYG | |
| | WEDMEVVSVSGNYCTDKKPAAINWIEGRGK | |
| | SVVAEATIPGDVVRKVLKSDVSALVELNIAKN | |
| | LVGSAMAGSVGGFNAHAANLVTAVFLALGQ | |
| | DPAQNVESSNCITLMKEVDGDLRISVSMPSIE | |
| | VGTIGGGTVLEPQGAMLDLLGVRGPHATAP | |
| | GTNARQLARIVACAVLAGELSLCAALAAGHL | |
| | VQSHMTHNRKPAEPTKPNNLDATDINRLKDG | |
| | SVTCIKS | |

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1.-35. (canceled)

36. A host cell comprising one or more vectors comprising a polynucleotide sequence encoding:
one or more genes of the mevalonate pathway; and one or more genes of the Nudix pathway.

37. The host cell according to any one of claim 36, wherein the host cell comprises two vectors, wherein
a. a first vector comprising a polynucleotide sequence encoding one or more genes of the mevalonate pathway; and
b. a second vector comprising a polynucleotide sequence encoding one or more genes of the Nudix pathway.

38. The host cell according to claim 1, wherein the one or more genes of the mevalonate pathway is selected from the group consisting of HMG-COA synthase (hmgS), acetoacetyl-CoA thiolase (atoB), HMG-COA reductase (hmgR), mevalonate kinase (mevk), phosphomevalonate kinase (pmk), mevalonate pyrophosphate decarboxylase (pmd) and (isopentenyl diphosphate) IPP isomerase (idi), and the one or more genes of the Nudix pathway is NUDX1 or NudI; optionally wherein the hmgR gene is truncated.

39. The host cell according to claim 37, wherein the second vector further comprises a polynucleotide sequence encoding one or more diphosphate synthase genes, prenyltransferase genes or combinations thereof in the host cell; optionally wherein the polynucleotide sequence in each of the vectors is operably linked to an inducible promoter, wherein the inducible promoter is a wild-type T7 RNA polymerase promoter or a variant of the wild-type T7 RNA polymerase promoter.

40. The host cell according to claim 37, wherein the first vector comprises
a. a polynucleotide sequence encoding the hmgS, atoB, hmgR genes of the mevalonate pathway operably linked to a first inducible promoter; and
b. a polynucleotide sequence encoding the mevk, pmk, pmd and idi genes of the mevalonate pathway operably linked to a second inducible promoter.

41. The host cell according to claim 39, wherein the second vector further comprises a polynucleotide sequence encoding a ribosomal binding site (RBS) that is located upstream of the polynucleotide sequence encoding the polynucleotide sequence encoding the Nudix pathway gene, the polynucleotide sequence encoding the diphosphate synthase gene or prenyltransferase gene, or combinations thereof; optionally wherein the polynucleotide sequence encoding the RBS in the second vector is selected from the group consisting of SEQ ID NO: 1, SEQ ID NO: 2, SEQ ID NO: 3, SEQ ID NO: 4 and SEQ ID NO: 5.

42. The host cell according to claim 39, wherein the diphosphate synthase or the prenyltransferase is a geranyl pyrophosphate synthase (GPPS) or a farnesyl diphosphate synthase (FPPS); optionally wherein the GPPS is isolated from Abies grandis (AgGPPS); optionally wherein the AgGPPS is truncated at the N-terminal; optionally wherein the truncated AgGPPS comprises the polypeptide sequence as set forth in SEQ ID NO: 7.

43. The host cell according to claim 42, wherein the FPPS is isolated from Saccharomyces cerevisiae or Escherichia coli; optionally wherein the FPPS is mutated at one or more amino acid positions; optionally wherein the mutated FPPS from Escherichia coli comprises the polypeptide sequence as set forth in SEQ ID NO. 9, and wherein the mutated FPPS from Saccharomyces cerevisiae comprises the polypeptide sequence as set forth in SEQ ID NO. 11, SEQ ID NO. 12, or SEQ ID NO. 13.

44. The host cell according to claim 37, wherein the NUDX1 is isolated from a plant; optionally wherein the NUDX1 is isolated from Rosa hybrida and has about 70%, 80%, 90% or 100% identity with the polypeptide sequence set forth in SEQ ID NO: 14, or wherein the NUDX1 is isolated from Arabidopsis thaliana and has about 70%, 80%, 90% or 100% identity with the polypeptide sequence set forth in SEQ ID NO: 15.

45. The host cell according to claim 37, wherein the NudI is isolated from a prokaryote.

46. The host cell according to claim 39, wherein the NUDX1 or NudI is co-expressed with the diphosphate synthase or the prenyltransferase.

47. The host cell according to claim 39, wherein the second vector further comprises one or more of:
a. a polynucleotide sequence encoding a geranyl synthase enzyme (GES) isolated from a plant, wherein the polynucleotide sequence encoding the GES is located upstream of the polynucleotide sequence encoding the diphosphate synthase or the prenyltransferase, and wherein the GES is co-expressed with the diphosphate enzyme or the prenyltransferase, and/or the nudix enzyme;
b. a polynucleotide sequence encoding a multiple antibiotic resistance protein (MarA) which is located downstream of the polynucleotide sequence encoding the diphosphate synthase or the prenyltransferase; or
c. a polynucleotide sequence encoding an alcohol acyltransferase (AAT) enzyme which is located downstream of the polynucleotide sequence encoding the diphosphate synthase or the prenyltransferase; or combinations thereof.

48. The host cell according to claim 36, wherein the host cell is deficient in the pta gene, the ackA gene or the ackA and pta genes.

49. The host cell according to claim 36, wherein the host cell is deficient in at least one gene involved in amino acid synthesis, oxidation of terpenoids, amino acid degradation or a combination thereof; optionally wherein the host cell is deficient in aroA, serC, yjgB and tnaA genes, or wherein the host cell is deficient in aroA, serC and tnaA genes.

50. The host cell according to claim 36, wherein the host cell is a bacterial cell; optionally wherein the bacterial cell is an Escherichia coli cell.

51. An engineered fusion protein comprising
a diphosphate synthase or prenyltransferase of the mevalonate pathway and a nudix hydrolase; or
a diphosphate synthase or prenyltransferase of the mevalonate pathway, a nudix hydrolase and a geranyl synthase enzyme (GES) of the terpene synthase pathway; or
a diphosphate synthase or prenyltransferase of the mevalonate pathway and a GES of the terpene synthase pathway.

52. The engineered fusion protein according to claim 51, wherein the diphosphate synthase or prenyltransferase is located downstream of the nudix hydrolase; optionally wherein the GES is located between the diphosphate synthase and the nudix hydrolase or between the prenyltransferase and the nudix hydrolase.

53. The engineered fusion protein according to claim 51, further comprising one or more linker sequences, wherein the one or more linker sequences is located between the nudix hydrolase and the diphosphate synthase or prenyltransferase of the fusion protein; wherein the linker is linked to the C-terminal of the nudix hydrolase and the N-terminal of the diphosphate synthase or prenyltransferase; and wherein the linker sequence comprises the sequence set forth in SEQ ID NO: 17 or SEQ ID NO: 18.

54. A method of geraniol, geranyl acetate, or geraniol and geranyl acetate production comprising culturing the host cell according to claim 36 in a culture medium, wherein the culture medium comprises an inducer and at least one carbon substrate.

55. The method according to claim 54, wherein the inducer is lactose or IPTG, and wherein the at least one carbon substrate is selected from the group consisting of glucose, glycerol, lactose, sucrose and combinations thereof.


SEQUENCE LISTING (66 sequences)

SEQ ID NO: 1 - DNA, 32 bp, synthetic construct
  source 1..32: mol_type = other DNA
gagatataataacgagataaggaaaagacaaa

SEQ ID NO: 2 - DNA, 35 bp, synthetic construct
  source 1..35: mol_type = other DNA
gccgagatataataacgagataaggaaaagacaaa

SEQ ID NO: 3 - DNA, 24 bp, synthetic construct
  source 1..24: mol_type = other DNA
tgccaactttaaaagaggcctaaa

SEQ ID NO: 4 - DNA, 30 bp, synthetic construct
  source 1..30: mol_type = other DNA
tttgtttaactttaagaaggagatatacat

SEQ ID NO: 5 - DNA, 30 bp, synthetic construct
  source 1..30: mol_type = other DNA
tttgtttaactttaacaaggagggatacat

SEQ ID NO: 6 - protein, 381 aa, Abies grandis
MAYSAMATMGYNGMAASCHTLHPTSPLKPFHGASTSLEAFNGEHMGLLRGYSKRKLSSYKNPASRSSNATVAQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 7 - protein, 296 aa, Abies grandis
MFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 8 - protein, 502 aa, Escherichia coli
MVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 9 - protein, 299 aa, Escherichia coli
MDFPQQLEACVKQANQALSRFIAPLPFQNTPVVETMQYGALLGGKRLRPFLVYATGHMFGVSTNTLDAPAAAVECIHAYFLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 10 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 11 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 12 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYWLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 13 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYWLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 14 - protein, 150 aa, Rosa hybrida
MGNETVVVAETAGSIKVAVVVCLLRGQNVLLGRRRSSLGDSTFSLPSGHLEFGESFEECAARELKEETDLDIGKIELLTVTNNLFLDEAKPSQYVAVFMRAVLADPRQEPQNIEPEFCDGWGWYEWDNLPKPLFWPLDNVVQDGFNPFPT

SEQ ID NO: 15 - protein, 147 aa, Arabidopsis thaliana
MSTGEAIPRVAVVVFILNGNSILLGRRRSSIGNSTFALPGGHLEFGESFEECAAREVMEETGLKIEKMKLLTVTNNVFKEAPTPSHYVSVSIRAVLVDPSQEPKNMEPEKCEGWDWYDWENLPKPLFWPLEKLFGSGFNPFTHGGGD

SEQ ID NO: 16 - DNA, 1518 bp, Ocimum basilicum
  source 1..1518: mol_type = genomic DNA
atgcagcacatggaggagagctcttccaagcgccgtgaatacttgctggaggaaacgactcgcaaactccagcgcaacgataccgaatccgtggaaaaactgaaactcattgataacatccagcaactgggcatcggttattacttcgaggacgcaattaacgctgtgctgcgttctccgttctctacaggtgaagaagatctgttcaccgccgctctgcgtttccgtctgctgcgtcacaacggcattgagatctccccggaaatcttcctgaagtttaaagacgaacgtggtaaattcgacgagtccgataccctcggcctgctgagcctgtacgaagcttccaacctgggtgttgcaggtgaagaaattctggaagaagcgatggaatttgccgaggcacgcctgcgccgctcgctgtccgaaccggccgcgccgctgcatggcgaagtggcgcaggctcttgacgtcccgcgccatctgcgtatggcgcgcctggaagcacgtcgctttatcgaacagtacggcaaacagtctgatcatgacggtgacctcctggaactggctattctggattataaccaggtgcaggcgcagcaccagtccgaactgactgaaatcatccgttggtggaaagagttgggacttgtcgataaactgtccttcggccgcgaccgtccgctggaatgtttcctgtggactgtaggcctgctgccagaaccgaagtactcctctgtacgtatcgagctcgctaaagcgatctccatcttattggtcattgatgatattttcgatacctacggtgaaatggatgatctgatcctgttcaccgatgctatccgtcgttgggatctggaagcgatggaaggtctgccggaatatatgaaaatctgctacatggccctgtataacaccacgaacgaagtttgctacaaagtactgcgtgataccggtcgtatcgtacttctgaacctcaaatccacatggattgacatgattgagggtttcatggaagaagcgaaatggttcaacggcggttcggcaccgaaactggaggagtacatcgaaaatggtgtgtctaccgcaggcgcatacatggcctttgcccacattttcttcctgattggcgaaggcgttacgcaccagaactcccagctgttcacccaaaaaccgtacccgaaagtgtttagcgccgccggtcgcatcctgcgtctgtgggatgatctgggcaccgcaaaagaagagcaggaacgtggtgatctggcgtcctgcgttcagctgtttatgaaggaaaaatccctgactgaagaagaagcgcgttctcgtatcctggaagaaattaaaggtctgtggcgcgatttaaacggcgaactggtgtacaacaaaaacctgccgctttccatcatcaaagttgcgctgaatatggcgcgcgcgtctcaagttgtatacaaacacgatcaggatacgtatttcagctccgtagataactacgtagacgctctgttttttactcagtga

SEQ ID NO: 17 - protein, 10 aa, synthetic construct
GGGGSGGPGS

SEQ ID NO: 18 - protein, 20 aa, synthetic construct
GGGGSGGGGSGGGGSGGPGS

SEQ ID NO: 19 - DNA, 1029 bp, Escherichia coli
  source 1..1029: mol_type = genomic DNA
atgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattaa

SEQ ID NO: 20 - DNA, 1476 bp, synthetic construct
  source 1..1476: mol_type = other DNA
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 21 - DNA, 1332 bp, synthetic construct
  source 1..1332: mol_type = other DNA
atgagcttaccgttcctgacttcggcaccgggcaaagttatcattttcggcgagcactctgctgtttacaacaaaccggcagttgcggcctccgtatctgcactgcgcacttatctgctgatctctgaaagctccgccccggatactattgaactggactttccggacatttcctttaaccacaaatggagcattaacgactttaacgcgatcactgaagatcaggtaaactcccagaaactggcaaaagcacagcaggctaccgatggtctgagccaggaactggtgtccctcctcgatcctttgctggctcaactctcggaatcgttccattaccatgctgctttctgttttctgtatatgtttgtttgcctctgcccgcacgcgaaaaacatcaaattctctctgaaatcgactctgccgattggtgccggcctgggttcgtccgcatctatttccgtttccctggcgctggccatggcctatctgggcggtctgatcggttccaacgacctggaaaaactctcggaaaacgataagcacatcgttaaccagtgggcgttcatcggtgaaaaatgtatccacggtaccccatccggtatcgataatgcggttgctacctacggtaacgcgttactgttcgaaaaagattctcataacggtactatcaacactaacaacttcaaatttttggacgattttccggcgattccgatgatcctgacttacacccgcatcccgcgtagcaccaaggatctggttgcacgcgttcgtgtactcgtgaccgaaaaattcccggaggttatgaaaccgatcctggatgcaatgggtgagtgcgcgctgcagggattagaaatcatgaccaaactgtcgaagtgtaaaggtacggacgacgaagctgttgaaacaaacaacgaactgtatgaacagctgctggaactgatccgtatcaaccacggcctgctggtcagcattggtgtgagccacccgggcctggaactgattaaaaatctttcggatgacctgcgcattggttctaccaagctgactggtgctggcggcggtggctgttctctgaccctcctgcgtcgcgatattacccaggaacaaatcgactcgttcaaaaaaaaactgcaggatgacttttcttatgaaaccttcgaaaccgacctgggcggtaccggctgttgtctcctgtccgccaaaaacttgaacaaagatctgaaaatcaaatctctcgtctttcagctgtttgaaaacaaaactaccaccaaacaacaaattgatgacctgctgctcccgggcaacaccaacttaccgtggacttcctaa

SEQ ID NO: 22 - DNA, 1356 bp, synthetic construct
  source 1..1356: mol_type = other DNA
atgtctgagcttcgcgctttctccgctccgggcaaggccctgctggccggaggctatctggtgctggacaccaaatatgaagcgtttgtagttggtctgtctgcccgtatgcatgcggtcgcgcacccgtatgggtcgctgcagggttcagataaattcgaggtgcgagtgaaaagcaaacagtttaaagacggtgagtggctgtatcacattagcccgaaatctggttttatcccggtatccatcggcggttccaaaaacccgttcattgaaaaagttattgccaacgttttctcctattttaaacctaacatggatgactactgtaaccgtaacctgttcgtgattgatattttctctgacgatgcttaccattcccaggaagacagcgttacggaacaccgtggcaaccgtcgtctgtcgtttcattcccaccgtatcgaagaagttccgaagactggcctgggtagctctgcaggcctggttaccgtcctgactactgctctggcctctttttttgtgtcggatctggaaaacaacgttgacaaatatcgtgaggtaattcataacctggctcaggtcgcacactgccaggcgcagggcaaaatcggctccggtttcgatgttgctgcggcagcttatggctccattcgttaccgtcgcttcccgcctgctctgatctcaaacctgccggatattggtagcgcaacctacggatcgaagctggctcacctggtggatgaggaagattggaatatcaccattaaatctaaccacctgccgtctggcctgaccctgtggatgggtgatatcaaaaacggctctgaaaccgtcaaactggtacagaaagttaagaattggtatgattctcacatgccggaatccctgaaaatctacaccgagctggatcacgcgaactcacgtttcatggacggtctgtccaaactggaccgtctgcacgaaacccacgatgattacagcgaccagatcttcgaatctctggaacgtaacgactgcacctgtcaaaaatacccggaaatcaccgaagttcgtgacgcggtagcgaccatccgccgctctttccgtaaaatcactaaggaaagcggcgctgacatcgaaccgccggttcagacctccctgctggacgattgccagactctgaaaggggtgctgacctgtctgattccgggtgcgggtggttatgacgctatcgcagtgatcacgaaacaggatgtagacctgcgtgcgcagactgcaaacgacaaacgttttagcaaagtacaatggctggatgttacccaggcggattggggtgttcgtaaagaaaaggaccctgaaacctacctggataaataa

SEQ ID NO: 23 - DNA, 1191 bp, synthetic construct
  source 1..1191: mol_type = other DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatcccgtatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcttagtcgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 24 - DNA, 573 bp, Escherichia coli
  source 1..573: mol_type = genomic DNA
atgcatcatcatcaccatcacgagctccaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 25 - DNA, 471 bp, Rosa hybrida
  source 1..471: mol_type = genomic DNA
atgcatcaccatcaccatcacggtaacgaaactgtggtagtcgcagaaaccgcgggtagcatcaaagttgccgttgtggtttgtctgttgcgtggtcaaaacgtgctcctgggtcgtcgtcgctcctctctgggtgacagcaccttctctctgccgagcggccacttagaatttggtgaatctttcgaagaatgtgccgcccgtgagctgaaagaagaaacggatctggacatcggtaaaatcgagctgctgaccgtaaccaacaacctgttcctggatgaagctaaaccgtcccagtatgttgcagtgttcatgcgtgctgttctggccgatccgcgtcaggagccgcagaacattgaacccgaattctgcgacggctggggttggtacgaatgggataacttaccgaagccactgttctggccgctcgacaacgtggtccaggatggcttcaacccgttcccgacttaa

SEQ ID NO: 26 - DNA, 426 bp, Escherichia coli
  source 1..426: mol_type = genomic DNA
gtgcgacaacggactattgtatgccctttgattcaaaatgatggtgcttatttgctgtgtaaaatggccgacgatcgcggcgttttccccggtcaatgggcgatttcgggtggcggcgtggagcctggcgaacgaattgaagaggcactacgccgcgaaattcgcgaagaactgggagaacagctgcttttgacagaaatcacgccgtggaccttcagcgatgatattcgcaccaagacgtatgcagatggtcgcaaggaagagatttatatgatttacctgatttttgactgcgtttctgccaaccgagaagtgaaaataaacgaagagtttcaggactacgcgtgggtaaaacctgaagatctggtgcattatgatttgaatgtcgccacccgaaaaacgttacgtttgaaaggtcttctgtaa

SEQ ID NO: 27 - protein, 502 aa, Saccharomyces cerevisiae; identical to SEQ ID NO: 8

SEQ ID NO: 28 - DNA, 1509 bp, synthetic construct
  source 1..1509: mol_type = other DNA
atggttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 29 - DNA, 43 bp, synthetic construct
  source 1..43: mol_type = other DNA
aaattaatacgactcactataggggaattgtgagcggataaca

SEQ ID NO: 30 - DNA, 43 bp, synthetic construct
  source 1..43: mol_type = other DNA
aaattaatacgactcactaatggggaattgtgagcggataaca

SEQ ID NO: 31 - DNA, 43 bp, synthetic construct
  source 1..43: mol_type = other DNA
aaattaatacgactcactcgaggggaattgtgagcggataaca

SEQ ID NO: 32 - DNA, 43 bp, synthetic construct
  source 1..43: mol_type = other DNA
aaattaatacgactcactataaaggaattgtgagcggataaca

SEQ ID NO: 33 - DNA, 43 bp, synthetic construct; identical to SEQ ID NO: 29
  source 1..43: mol_type = other DNA

SEQ ID NO: 34 - DNA, 43 bp, synthetic construct
  source 1..43: mol_type = other DNA
aaattaatacgactcactacagacgaattgtgagcggataaca

SEQ ID NO: 35 - DNA, 43 bp, synthetic construct
  source 1..43: mol_type = other DNA
aaattaatacgactcactagcggagaattgtgagcggataaca

SEQ ID NO: 36 - DNA, 43 bp, synthetic construct
  source 1..43: mol_type = other DNA
aaattaatacgactcactaacaccgaattgtgagcggataaca

SEQ ID NO: 37 - DNA, 60 bp, synthetic construct
  source 1..60: mol_type = other DNA
ggtagtggcggcggtggcagcggtggcccgggcagcatgcatcatcatcaccatcacgag

SEQ ID NO: 38 - DNA, 60 bp, synthetic construct
  source 1..60: mol_type = other DNA
caccgccgccactaccaccgccgccgctgccaccgccaccagtcgggaacgggttgaagc

SEQ ID NO: 39 - DNA, 41 bp, synthetic construct
  source 1..41: mol_type = other DNA
gcggtggcccgggcagcatgcatcatcatcaccatcacgag

SEQ ID NO: 40 - DNA, 47 bp, synthetic construct
  source 1..47: mol_type = other DNA
gcccgggccaccgctgccaccgccaccagtcgggaacgggttgaagc

SEQ ID NO: 41 - DNA, 20 bp, synthetic construct
  source 1..20: mol_type = other DNA
tatgccgcaaaagaagcggg

SEQ ID NO: 42 - DNA, 20 bp, synthetic construct
  source 1..20: mol_type = other DNA
cacgaatgcggaacggttca

SEQ ID NO: 43 - DNA, 32 bp, synthetic construct; identical to SEQ ID NO: 1
  source 1..32: mol_type = other DNA

SEQ ID NO: 44 - DNA, 40 bp, synthetic construct
  source 1..40: mol_type = other DNA
atgttcgatttcaacaaatacatggacagcaaagccatga

SEQ ID NO: 45 - DNA, 35 bp, synthetic construct; identical to SEQ ID NO: 2
  source 1..35: mol_type = other DNA

SEQ ID NO: 46 - DNA, 40 bp, synthetic construct; identical to SEQ ID NO: 44
  source 1..40: mol_type = other DNA

SEQ ID NO: 47 - DNA, 24 bp, synthetic construct
  source 1..24: mol_type = other DNA
tgccaactttaaaagaggcctaaa

SEQ ID NO: 48 - DNA, 40 bp, synthetic construct
  source 1..40: mol_type = other DNA
atggcttcagaaaaagaaattaggagagagagattcttga

SEQ ID NO: 49 - DNA, 35 bp, synthetic construct; identical to SEQ ID NO: 2
  source 1..35: mol_type = other DNA

SEQ ID NO: 50 - DNA, 40 bp, synthetic construct; identical to SEQ ID NO: 48
  source 1..40: mol_type = other DNA

SEQ ID NO: 51 - DNA, 24 bp, synthetic construct
  source 1..24: mol_type = other DNA
tgccaactttaaaagaggcctaaa

SEQ ID NO: 52 - DNA, 40 bp, synthetic construct; identical to SEQ ID NO: 48
  source 1..40: mol_type = other DNA

SEQ ID NO: 53 - DNA, 35 bp, synthetic construct; identical to SEQ ID NO: 2
  source 1..35: mol_type = other DNA

SEQ ID NO: 54 - DNA, 40 bp, synthetic construct; identical to SEQ ID NO: 48
  source 1..40: mol_type = other DNA

SEQ ID NO: 55 - DNA, 30 bp, synthetic construct; identical to SEQ ID NO: 4
  source 1..30: mol_type = other DNA

SEQ ID NO: 56 - DNA, 40 bp, synthetic construct
  source 1..40: mol_type = other DNA
atgcatcatcatcaccatcacgagctcgactttccgcagc

SEQ ID NO: 57 - DNA, 30 bp, synthetic construct; identical to SEQ ID NO: 5
  source 1..30: mol_type = other DNA

SEQ ID NO: 58 - DNA, 40 bp, synthetic construct; identical to SEQ ID NO: 56
  source 1..40: mol_type = other DNA

SEQ ID NO: 59 - DNA, 30 bp, synthetic construct; identical to SEQ ID NO: 4
  source 1..30: mol_type = other DNA

SEQ ID NO: 60 - DNA, 30 bp, synthetic construct
  source 1..30: mol_type = other DNA
atcgattcacaacacgttgatgaagtgatt

SEQ ID NO: 61 - DNA, 22 bp, synthetic construct
  source 1..22: mol_type = other DNA
ctcgagaataaggaggtaagtc

SEQ ID NO: 62 - DNA, 22 bp, synthetic construct
  source 1..22: mol_type = other DNA
aacacaaacagaggggaaaaaa

SEQ ID NO: 63 - DNA, 30 bp, synthetic construct
  source 1..30: mol_type = other DNA
tcactagacacatcggataaggaggtacct

SEQ ID NO: 64 - DNA, 32 bp, synthetic construct
  source 1..32: mol_type = other DNA
tccttatcattccatcccataagagaggcact

SEQ ID NO: 65 - DNA, 31 bp, synthetic construct
  source 1..31: mol_type = other DNA
ccccactcaggaaaataataagagaggacct

SEQ ID NO: 66 - protein, 1054 aa, Saccharomyces cerevisiae
MPPLFKGLKQMAKPIAYVSRFSAKRPIHIILFSLIISAFAYLSVIQYYFNGWQLDSNSVFETAPNKDSNTLFQECSHYYRDSSLDGWVSITAHEASELPAPHHYYLLNLNFNSPNETDSIPELANTVFEKDNTKYILQEDLSVSKEISSTDGTKWRLRSDRKSLFDVKTLAYSLYDVFSENVTQADPFDVLIMVTAYLMMFYTIFGLFNDMRKTGSNFWLSASTVVNSASSLFLALYVTQCILGKEVSALTLFEGLPFIVVVVGFKHKIKIAQYALEKFERVGLSKRITTDEIVFESVSEEGGRLIQDHLLCIFAFIGCSMYAHQLKTLTNFCILSAFILIFELILTPTFYSAILALRLEMNVIHRSTIIKQTLEEDGVVPSTARIISKAEKKSVSSFLNLSVVVIIMKLSVILLFVFINFYNFGANWVNDAFNSLYFDKERVSLPDFITSNASENFKEQAIVSVTPLLYYKPIKSYQRIEDMVLLLLRNVSVAIRDRFVSKLVLSALVCSAVINVYLLNAARIHTSYTADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS